(19) United States
(12) Patent Application Publication
(10) Pub. No.: US 2012/0255051 A1
(43) Pub. Date: Oct. 4, 2012

(54) HERBICIDE TOLERANT PLANTS

(76) Inventors: Rene Ruiter, Heusden (NL); Timothy Golds, Merelbeke (GB)

(21) Appl. No.: 13/514,151

(22) PCT Filed: Dec. 6, 2010
(86) PCT No.: PCT/EP10/07483

§ 371 Date: Jun. 14, 2012

Related U.S. Application Data
(60) Provisional application No. 61/288,958, filed on Dec. 22, 2009.

(30) Foreign Application Priority Data
Dec. 22, 2009 (EP) 09015848.6

(51) Int. Cl. C12N 15/54; A01H 5/10; A01P 13/00; A01H 1/02; A01H 1/06; A01N 47/38; A01H 5/00; C12N 5/10

(52) U.S. Cl. 800/260; 800/300; 435/419; 536/23.2; 800/278; 504/273

(57) ABSTRACT

The present invention relates to Brassica plants comprising full knockout AHAS alleles and to brassica plant comprising a combination of full knockout AHAS alleles and AHAS alleles encoding herbicide tolerant AHAS proteins, nucleic acid sequences representing full knockout AHAS alleles, as well as methods for generating and identifying said plants and alleles, which can be used to obtain herbicide tolerant plants.

### FIELD OF THE INVENTION

This invention relates to crop plants and parts, particularly plants of the Brassicaceae family, in particular Brassica species, which are tolerant to herbicides, more specifically AHAS-inhibiting herbicides. This invention also relates to mutant AHAS nucleic acids representing full knockout AHAS alleles. More particularly, this invention relates to nucleic acids representing full knockout and mutant AHAS proteins that affect tolerance to AHAS-inhibiting herbicides in plants.

### BACKGROUND OF THE INVENTION

Acetohydroxyacid synthase (AHAS; EC 4.1.3.18, also known as acetolactate synthase or ALS), is a critical enzyme for the biosynthesis of branched chain amino acids in plants (Tan et al., 2005, Pest Manag Sci, 61:246-257). AHAS is the site of action of several structurally diverse herbicide families, including sulfonylureas, imidazolinones, sulfonylaminocarbonyltriazolinones, the triazolopyrimidines and the pyrimidyl(oxy/thio)benzoates. Since AHAS is not present in animals AHAS-inhibiting herbicides display very low toxicity in animals (Duggleby et al., 2008, Plant Physiology and Biochemistry 46, 309-324).

Brassica napus is allotetraploid, having an A and a C genome, and comprises five AHAS loci. AHAS2, AHAS3 and AHAS4 originate from the A genome, whereas AHAS1 and AHAS5 originate from the C genome. AHAS1 and AHAS3 are the only genes that are constitutively expressed and encode the primary AHAS activities essential to growth and development in B. napus (Tan et al., Pest Manag Sci 61, p246-257, 2005).

Various plants with mutations in AHAS that confer tolerance to one or more AHAS-inhibiting herbicides have been described (for an overview, see Duggleby, et al., 2008, table 2, which is incorporated herein by reference). For instance, mutation of Pro197 to e.g. Ser, Leu, His Thr, Gln, Ala or Thr can confer tolerance to SU, IMI, PC, TP and/or SACT and has been described in various plant species including Arabidopsis thaliana, pigweed, wild radish, crown daisy, tobacco and canola (Haugh et al., 1988 Mol Gen Genet 211: 266-271; Sibony et al., Weed Res 41:509-522, 2001; Yu et al., 2003, Weed Science, 51(6), p. 831-838; Tal and Rubin 2004, Resistant Pest Management Newsletter. 13: p31-33; Lee et al., 1988, EMBO J. 7(5):p1241-1248; Ruiter et al., 2003, Plant Mol. Biol. 53(5): p675-89; Shimizu et al., 2008, Plant Physiol. 147(4): p1976-83)

Oilseed rape imidazolinone-tolerant mutants PM1 and PM2, currently marketed as Clearfield® canola, display single nucleotide substitutions resulting in an asparagine to serine substitution at amino acid position 653 in the AHAS1 protein (PM1) and a tryptophan to leucine substitution at amino acid position 574 in the AHAS3 protein (PM2). PM1 is tolerant to imidazolinones only, but PM2 is crosstolerant to both imidazolinones and sulfonylureas, whereby the imidazolinones-tolerance level contributed by PM2 is much higher than that from PM1. The highest level of tolerance to imidazolinone herbicides is obtained when PM1 and PM2 mutations are stacked and homozygous (Tan et al., 2005).

WO09/046,334 describes mutated acetohydroxyacid synthase (AHAS) nucleic acids and the proteins encoded by the mutated nucleic acids, as well as canola plants, cells, and seeds comprising the mutated genes, whereby the plants display increased tolerance to imidazolinones and sulfonylureas.

WO09/031,031 discloses herbicide-resistant Brassica plants and novel polynucleotide sequences that encode wild-type and imidazolinone-resistant Brassica acetohydroxyacid synthase large subunit proteins, seeds, and methods using such plants.

U.S. patent application Ser. No. 09/001,3424 describes improved imidazolinone herbicide resistant Brassica lines, including Brassica juncea, methods for generation of such lines, and methods for selection of such lines, as well as Brassica AHAS genes and sequences and a gene allele bearing a point mutation that gives rise to imidazolinone herbicide resistance.

WO08/124,495 discloses nucleic acids encoding mutants of the acetohydroxyacid synthase (AHAS) large subunit comprising at least two mutations, for example double and triple mutants, which are useful for producing transgenic or non-transgenic plants with improved levels of tolerance to AHAS-inhibiting herbicides. The invention also provides expression vectors, cells, plants comprising the polynucleotides encoding the AHAS large subunit double and triple mutants, plants comprising two or more AHAS large subunit single mutant polypeptides, and methods for making and using the same.

Nevertheless, further improvement of tolerance to AHAS-inhibiting herbicides in crop plants, particularly oilseed rape plants is desirable.

This invention makes a significant contribution to the art by providing herbicide tolerant plants comprising a combination of AHAS alleles representing full knockout alleles and AHAS alleles encoding herbicide tolerant AHAS proteins. By combining herbicide tolerant AHAS alleles with full knockout AHAS alleles, the invention provides an alternative approach to obtain efficient tolerance to AHAS-inhibiting herbicides in crop plants, particularly oilseed rape plants.

This problem is solved as herein after described in the different embodiments, examples and claims.

### SUMMARY OF THE INVENTION

In a first embodiment the invention provides a Brassica plant comprising a full knockout AHAS allele. A full knockout AHAS allele refers to a nucleic acid sequence of an AHAS gene, which encodes no functional AHAS protein, i.e. an AHAS protein that does not participate nor influence AHAS dimer formation, or no AHAS protein at all.

In another embodiment, invention provides a Brassica plant wherein the full knockout AHAS allele comprises a nonsense mutation, which is a mutation in a AHAS allele whereby one or more translation stop codons are introduced into the coding DNA and the corresponding mRNA sequence of the corresponding wild type AHAS allele, whereby the stop codon results in the production of no functional AHAS protein.

In yet another embodiment, invention provides a Brassica plant wherein the full knockout AHAS allele is selected from the group consisting of:
a) a nucleotide sequence comprising a stop codon at a position corresponding to nt 871-873 of SEQ ID NO: 1 or nt 826-828 of SEQ ID NO: 3;
b) a nucleotide sequence comprising a stop codon at a position corresponding to nt 862-864 of SEQ ID NO: 1 or nt 808-810 of SEQ ID NO: 5;
c) a nucleotide sequence comprising a stop codon at a position corresponding to nt 775-777 of SEQ ID NO: 1 or nt 721-723 of SEQ ID NO: 5; or
d) a nucleotide sequence comprising a stop codon at a position corresponding to nt 799-801 of SEQ ID NO: 1 or nt 745-747 of SEQ ID NO: 5.

The invention also provides a Brassica plant further comprising in its genome at least one second mutant AHAS allele, said second mutant AHAS allele encoding a herbicide tolerant AHAS protein.

In another embodiment, the herbicide tolerant AHAS protein comprises a serine at a position corresponding to position 197 of SEQ ID NO: 2, or position 182 of SEQ ID NO: 4 or position 179 of SEQ ID NO: 6. Alternatively, the herbicide tolerant AHAS protein comprises at least two amino acid substitutions.

In yet another embodiment, the herbicide tolerant AHAS protein(s) comprise(s) an amino acid sequence having at least 75%, at least 80%, at least 85%, at least 90%, at least 95%, 98%, 99% or 100% sequence identity to SEQ ID NO: 2, SEQ ID NO: 4 or SEQ ID NO: 6.

In a further embodiment, the AHAS allele(s) of the invention comprise(s) a nucleotide sequence having at least 75%, at least 80%, at least 85%, at least 90%, at least 95%, 98%, 99% or 100% sequence identity to SEQ ID NO: 1, SEQ ID NO: 3 or SEQ ID NO: 5.

It is also an embodiment of the invention to provide plant cells, gametes, seeds, embryos, either zygotic or somatic, progeny or hybrids of plants containing the mutant AHAS alleles of the invention.

The invention further provides Brassica seeds selected from the group consisting of:
a) Brassica seed comprising AHAS1-HETO112 having been deposited at the NCIMB Limited on Dec. 17, 2009, under accession number NCIMB 41690;
b) Brassica seed comprising AHAS3-HETO102 having been deposited at the NCIMB Limited on Dec. 17, 2009, under accession number NCIMB 41687;
c) Brassica seed comprising AHAS3-HETO103 having been deposited at the NCIMB Limited on Dec. 17, 2009, under accession number NCIMB 41688; or
d) Brassica seed comprising AHAS3-HETO104 having been deposited at the NCIMB Limited on Dec. 17, 2009, under accession number NCIMB 41689;
   Also provided are a Brassica plant, or a cell, part, seed or progeny thereof, obtained from the above described seeds.

In one embodiment, nucleic acid sequences representing full knockout AHAS alleles as described above are provided.

The invention also provides a method for transferring at least one selected full knockout AHAS allele of the invention from one plant to another plant comprising the steps of:
e) identifying a first plant comprising at least one selected full knockout AHAS allele or generating a first plant comprising at least one selected full knockout AHAS allele;
f) crossing the first plant with a second plant not comprising the at least one selected full knockout AHAS allele and collecting F1 hybrid seeds from the cross,
g) optionally, identifying F1 plants comprising the at least one selected full knockout AHAS allele;
h) backcrossing F1 plants comprising the at least one selected full knockout AHAS allele with the second plant not comprising the at least one selected full knockout AHAS allele for at least one generation (x) and collecting BCx seeds from the crosses; and
i) identifying in every generation BCx plants comprising the at least one selected full knockout AHAS allele.

The invention further provides a method for combining a full knockout AHAS allele of the invention with a herbicide tolerant AHAS allele in one plant comprising the steps of:
j) generating and/or identifying at least one plant comprising at least one selected full knockout AHAS allele and at least one plant comprising at least one selected herbicide tolerant AHAS allele;
k) crossing the at least two plants and collecting F1 hybrid seeds from the at least one cross; and
l) optionally, identifying an F1 plant comprising at least one selected full knockout AHAS allele and the at least one selected herbicide tolerant AHAS allele.

In another embodiment, methods are provided for producing the plant as described above, as well as methods to increase the herbicide tolerance of a plant plant by combining at least one full knockout AHAS allele of the invention and at least one herbicide tolerant AHAS allele in the genomic DNA of the plant.

The invention further provides methods for controlling weeds in the vicinity of crop plants, as well as methods for treating plants comprising a combination of full knockout and herbicide tolerant AHAS alleles with on or more AHAS-inhibiting herbicides.

The invention also relates to the use of a full knockout AHAS allele of the invention to obtain a herbicide tolerant plant.

In yet another embodiment, the invention relates to the use of a plant of the invention to produce seed comprising one or more full knockout AHAS alleles or to produce a crop of oilseed rape, comprising one or more full knockout AHAS alleles.

### BRIEF DESCRIPTION OF THE DRAWINGS

FIG. 1: Multiple sequence alignment of the amino acid sequences of B. napus AHAS1 (BN1), B. napus AHAS3 (BN3) and A. thaliana AHAS (AT) proteins from GenBank CAA77613.1, CAA77615.1 and AY042819.1, respectively

FIG. 2: The effect of combining AHAS full knockouts with AHAS missense alleles on tolerance to thiencarbazone-methyl pre-planting application in the greenhouse. A. AHAS1 missense allele (HETO108) combined with AHAS3 missense allele (HETO111). From left to right: HETO108/HETO108 HETO111/HETO111 untreated; HETO108/HETO108 HETO111/HETO111 treated; HETO108/HETO108 AHAS3 wt/AHAS3 wt treated; AHAS1 wt/AHAS1 wt HETO111/HETO111 treated; AHAS1 wt/AHAS1 wt AHAS3 wt/AHAS3 wt treated. B. AHAS1 knock-out allele (HETO112) combined with AHAS3 missense allele (HETO111). From left to right: HETO112/HETO112 HETO111/HETO111 untreated; HETO112/HETO112 HETO111/HETO111 treated; HETO112/HETO112 AHAS3 wt/AHAS3 wt treated; AHAS1 wt/AHAS1 wt HETO111/HETO111 treated; AHAS1 wt/AHAS1 wt AHAS3 wt/AHAS3 wt treated. C. AHAS1 missense allele (HETO108) combined with AHAS3 knock-out allele (HETO104). From left to right: HETO108/HETO108 HETO104/HETO104 untreated; HETO108/HETO108 HETO104/HETO104 treated; HETO108/HETO108 AHAS3 wt/AHAS3 wt treated; AHAS1 wt/AHAS1 wt HETO104/HETO104 treated; AHAS1 wt/AHAS1 wt AHAS3 wt/AHAS3 wt treated. Wt=wild-type.

FIG. 3: The effect of combining AHAS full knockouts with AHAS missense alleles on tolerance to thiencarbazone-methyl post-emergence spraying in the greenhouse. A. AHAS1 missense allele (HETO108) combined with AHAS3 missense allele (HETO111). From left to right: Elite parent line untreated; HETO108/HETO108 HETO111/HETO111 treated; HETO108/HETO108 AHAS3 wt/AHAS3 wt treated; AHAS1 wt/AHAS1 wt HETO111/HETO111 treated; AHAS1 wt/AHAS1 wt AHAS3 wt/AHAS3 wt treated. B. AHAS1 knock-out allele (HETO112) combined with AHAS3 missense allele (HETO111). From left to right: Elite parent line untreated; HETO112/HETO112 HETO111/HETO111 treated; HETO112/HETO112 AHAS3 wt/AHAS3 wt treated; AHAS1 wt/AHAS1 wt HETO111/HETO111 treated; AHAS1 wt/AHAS1 wt AHAS3 wt/AHAS3 wt treated. C. AHAS1 missense allele (HETO108) combined with AHAS3 knock-out allele (HETO104). From left to right: Elite parent line untreated; HETO108/HETO108 HETO104/HETO104 treated; HETO108/HETO108 AHAS3 wt/AHAS3 wt treated; AHAS1 wt/AHAS1 wt HETO104/HETO104 treated; AHAS1 wt/AHAS1 wt AHAS3 wt/AHAS3 wt treated. Wt=wild-type.

### GENERAL DEFINITIONS

The term “nucleic acid sequence” (or nucleic acid molecule) refers to a DNA or RNA molecule in single or double stranded form, particularly a DNA encoding a protein or protein fragment according to the invention. An “endogenous nucleic acid sequence” refers to a nucleic acid sequence which is within a plant cell, e.g. an endogenous allele of an AHAS gene present within the nuclear genome of a Brassica cell. An “isolated nucleic acid sequence” is used to refer to a nucleic acid sequence that is no longer in its natural environment, for example in vitro or in a recombinant host cell such as a bacteria or plant.

The term “gene” means a DNA sequence comprising a region (transcribed region), which is transcribed into an RNA molecule (e.g. a pre-mRNA, comprising intron sequences, which is then spliced into a mature mRNA) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. “Endogenous gene” is used to differentiate from a “foreign gene”, “transgene” or “chimeric gene”, and refers to a gene from a plant of a certain plant genus, species or variety, which has not been introduced into that plant by transformation (i.e. it is not a ‘transgene’), but which is normally present in plants of that genus, species or variety, or which is introduced in that plant from plants of another plant genus, species or variety, in which it is normally present, by normal breeding techniques or by somatic hybridization, e.g., by protoplast fusion. Similarly, an “endogenous allele” of a gene is not introduced into a plant or plant tissue by plant transformation, but is, for example, generated by plant mutagenesis and/or selection or obtained by screening natural populations of plants.

The terms “protein” or “polypeptide” are used interchangeably and refer to molecules consisting of a chain of amino acids, without reference to a specific mode of action, size, 3-dimensional structure or origin. A “fragment” or “portion” of an AHAS protein may thus still be referred to as a “protein”. An “isolated protein” is used to refer to a protein which is no longer in its natural environment, for example in vitro or in a recombinant bacterial or plant host cell. An “enzyme” is a protein or protein complex comprising enzymatic activity, such as functional AHAS enzymes.

As used herein “AHAS protein”, refers to the protein(s) or polypeptide(s) constituting the catalytic subunit of the AHAS enzyme, which is involved in the biosynthesis of branched chain amino acids, also known as “acetohydroxyacid synthase” or “acetolactate synthase”. In plants and microorganisms, the carbon skeletons of these amino acids are synthesized from pyruvate alone (valine synthesis), pyruvate plus acetyl-CoA (leucine) or pyruvate plus 2-ketobutyrate (isoleucine). The first step in this process, in which either 2-acetolactate (AL) or 2-aceto-2-hydroxybutyrate (AHB) is formed, is catalyzed by acetohydroxyacid synthase (AHAS, EC 2.2.1.6). The AHAS enzyme is composed of two subunits; a catalytic subunit and a regulatory subunit, also referred to as the large and the small subunit respectively. The catalytic subunit has a molecular mass in the 59-66 kDa range and in eukaryotes it is synthesized as a larger precursor protein having an N-terminal peptide which is required to direct the protein to mitochondria in fungi and to chloroplasts in plants. The regulatory subunit possesses no AHAS activity but greatly stimulates the activity of the catalytic subunit. It is over 50 kDa in plants and is also synthesized as a larger precursor protein with an N-terminal organelle-targeting peptide. Gel in filtration studies indicated that in solution the catalytic subunit of Arabidopsis thaliana AHAS exists as a dimer. However, in the presence of any of the sulfonylurea herbicides it crystallizes as a tetramer, and the molecular mass of the complex between the regulatory and catalytic subunits also suggests the presence of four of each subunit in the assembly. Each tetramer of the catalytic subunit of A. thaliana AHAS has four active sites. Each active site is at the interface of two monomers; hence the minimal requirement for AHAS activity is a dimer of the catalytic subunits. The biological relevance of the tetramers is unclear; they may (Duggleby et al., 2008). The amino acid sequence of the AHAS protein from A. thaliana, the AHAS1 and AHAS3 protein from B. napus are represented in the sequence listing in SEQ ID NO: 2, SEQ ID NO: 4 and SEQ ID NO: 6 respectively.

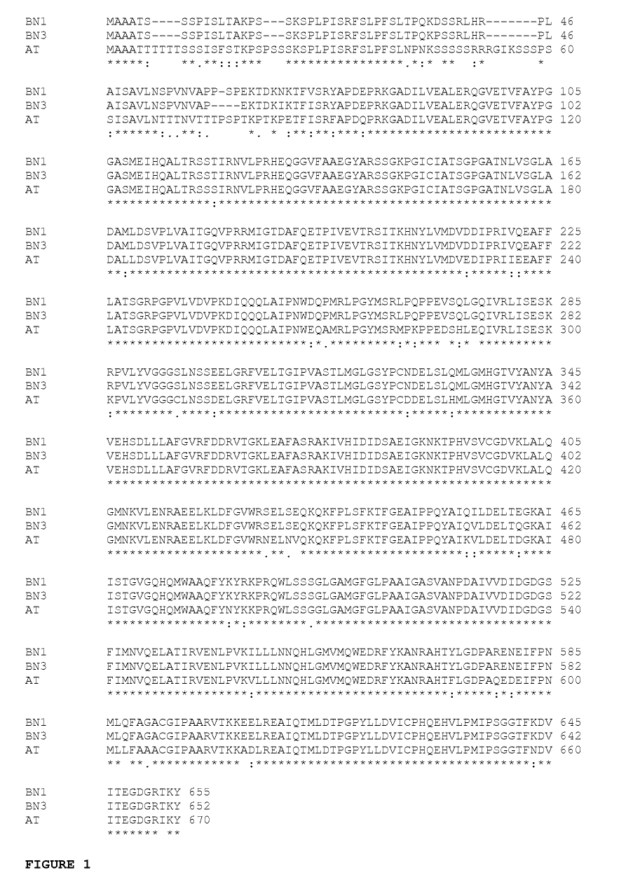

In A. thaliana, the AHAS protein (GenBank: CAB62345.1, AAM92569.1 and AY042819.1) is synthesized as a 663 amino acids (aa) long precursor, while the mature protein without the chloroplast transit peptide starts at aa 98. In B. napus, the AHAS1 (GenBank: CAA77613.1) and AHAS3 (GenBank: CAA77615.1) precursor proteins are 655 and 652 aa long, with the mature proteins starting at aa 83 and 80 respectively. Each polypeptide of A. thaliana AHAS consists of three domains, α (residues 86-280), β (residues 281-451) and γ (residues 463-639) with each having a similar overall fold of a six-stranded parallel b-sheet surrounded by six to nine helices. Residues involved in forming the dimer interface in A. thaliana are located between aa 119-217 and between aa 508-607. In B. napus these are respectively located between aa 104-202 and between aa 493-592 (AHAS1), and between aa 101-199 and between aa 490-589 (AHAS3). An alignment of the amino acid sequences of A. thaliana and B. napus AHAS proteins is represented in FIG. 1. In tobacco, the residues M542 and H142 appear to be involved in stabilization of the tertiary structure and dimer interaction (Le et al., 2004, Biochem Biophys Res Commun. 7; 317(3), p930-938). Also, the regions between aa 567-582 and the region C-terminal of aa 630 of the Tobacco AHAS protein were found be involved in the binding/stabilization of the active dimer, as deletion of these domains resulted in monomer formation (Kim et al., 2004, Biochem J. 15; 384, p 59-68.).

The term “AHAS gene” refers herein to the nucleic acid sequence encoding an acetohydroxyacid synthase catalytic subunit protein (i.e. an AHAS protein). The AHAS gene is intronles (Mazur et al., 1987, Plant Physiol., December; 85, p1110-1117.). Sequences of genes/coding sequences of A. thaliana AHAS (GenBank AY042819) and B. napus AHAS1 and AHAS3 are represented in the sequence listing in SEQ ID NO:1, SEQ ID NO: 3 and SEQ ID NO: 5 respectively.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent. In amphidiploid species, essentially two sets of diploid genomes exist, whereby the chromosomes of the two genomes are referred to as “homeologous chromosomes” (and similarly, the loci or genes of the two genomes are referred to as homeologous loci or genes). A diploid, or amphidiploid, plant species may comprise a large number of different alleles at a particular locus.

As used herein, the term “heterozygous” means a genetic condition existing when two different alleles reside at a specific locus, but are positioned individually on corresponding pairs of homologous chromosomes in the cell. Conversely, as used herein, the term “homozygous” means a genetic condition existing when two identical alleles reside at a specific locus, but are positioned individually on corresponding pairs of homologous chromosomes in the cell.

As used herein, the term “locus” (loci plural) means a specific place or places or a site on a chromosome where for example a gene or genetic marker is found. For example, the “AHAS1 locus” refers to the position on a chromosome where the AHAS1 gene (and two AHAS1 alleles) may be found, while the “AHAS3 locus” refers to the position on a chromosome where the AHAS3 gene (and two AHAS3 alleles) may be found.

“Essentially similar”, as used herein, refers to sequences having at least 75%, at least 80%, at least 85%, at least 90%, at least 95%, 98%, 99% or 100% sequence identity. These nucleic acid sequences may also be referred to as being “substantially identical” or “essentially identical” to the AHAS sequences provided in the sequence listing. The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

The term “ortholog” of a gene or protein refers herein to the homologous gene or protein found in another species, which has the same function as the gene or protein, but is (usually) diverged in sequence from the time point on when the species harboring the genes diverged (i.e. the genes evolved from a common ancestor by speciation). Orthologs of the Brassica napus AHAS genes may thus be identified in other plant species (e.g. Brassica juncea, etc.) based on both sequence comparisons (e.g. based on percentages sequence identity over the entire sequence or over specific domains) and/or functional analysis.

The term “mutant” or “mutation” refers to e.g. a plant or gene that is different from the so-called “wild type” variant (also written “wildtype” or “wild-type”), which refers to a typical form of e.g. a plant or gene as it most commonly occurs in nature. A “wild type plant” refers to a plant with the most common phenotype of such plant in the natural population. A “wild type allele” refers to an allele of a gene required to produce the wild-type phenotype. A mutant plant or allele can occur in the natural population or be produced by human intervention, e.g. by mutagenesis, and a “mutant allele” thus refers to an allele of a gene required to produce the mutant phenotype. As used herein, the term “mutant AHAS allele” (e.g. mutant AHAS1 or AHAS3) refers to an AHAS allele, which differs from the wildtype AHAS allele at one or more nucleotide positions, i.e. it comprises one or more mutations in its nucleic acid sequence when compared to the wild type allele.

Mutations in nucleic acid sequences may include for instance:

(a) a “missense mutation”, which is a change in the nucleic acid sequence that results in the substitution of an amino acid for another amino acid;
(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
(c) an “insertion mutation” of one or more amino acids, due to one or more codons having been added in the coding sequence of the nucleic acid;
(d) a “deletion mutation” of one or more amino acids, due to one or more codons having been deleted in the coding sequence of the nucleic acid;
(e) a “frameshift mutation”, resulting in the nucleic acid sequence being translated in a different frame downstream of the mutation. A frameshift mutation can have various causes, such as the insertion, deletion or duplication of one or more nucleotides, but also mutations which affect pre-mRNA splicing (splice site mutations) can result in frameshifts;
(f) a “splice site mutation”, which alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered through incorrect splicing, or one or more introns may be retained, or alternate splice donors or acceptors may be generated, or splicing may be initiated at an alternate position (e.g. within an intron), or alternate polyadenylation signals may be generated. Correct pre-mRNA splicing is a complex process, which can be affected by various mutations in the nucleotide sequence a genes. In higher eukaryotes, such as plants, the major spliceosome splices introns containing GU at the 5′ splice site (donor site) and AG at the 3′ splice site (acceptor site). This GU-AG rule (or GT-AG rule; see Lewin, Genes VI, Oxford University Press 1998, pp 885-920, ISBN 0198577788) is followed in about 99% of splice sites of nuclear eukaryotic genes, while introns containing other dinucleotides at the 5′ and 3′ splice site, such as GC-AG and AU-AC account for only about 1% and 0.1% respectively

As used herein, a “full knock-out allele” is a mutant allele directing a significantly reduced or no functional AHAS expression, i.e. a significantly reduced amount of functional AHAS protein or no functional AHAS protein, in the cell in vivo. Basically, any mutation which results in a protein comprising at least one amino acid insertion, deletion and/or substitution relative to the wild type protein can lead to significantly reduced or no enzymatic activity. It is, however, understood that mutations in certain parts of the protein encoding sequence are more likely to result in a reduced function of the mutant AHAS protein, such as mutations leading to truncated proteins, whereby significant portions of the functional and/or structural domains, are lacking.

To determine whether a mutant AHAS allele is a full knock-out allele, it can be analyzed whether that specific allele is indeed not or significantly less expressed at the mRNA and/or protein level, and in case it still is expressed, whether the molecular mass of the protein indicates multimer or monomer formation, as for instance described Kim et al. (Biochem J. 15; 384, p 59-68, 2004). Alternatively, crosses can be performed on e.g. plants, for which AHAS function is essential, whereby (double) homozygous for the mutant allele are expected to be obtained, and if these are not recovered, the mutant allele functions as a knockout allele, as for instance described herein below.

As used herein, a “significantly reduced amount of functional AHAS protein” (e.g. functional AHAS1 or AHAS2 protein) refers to a reduction in the amount of a functional AHAS protein produced by the cell comprising a full knockout AHAS allele by at least 30%, 40%, 50%, 60%, 70%, 80%, 90%, 95% or 100% (i.e. no functional protein is produced by the cell) as compared to the amount of the functional AHAS protein produced by the cell not comprising the a full knockout AHAS allele. This definition encompasses the production of a “non-functional”AHAS protein (e.g. truncated AHAS protein) having no biological activity in vivo, the reduction in the absolute amount of the functional AHAS protein (e.g. no functional AHAS protein being made due to the mutation in the AHAS gene) and/or the production of an AHAS protein with significantly reduced biological activity compared to the activity of a functional wild type AHAS protein (such as an AHAS protein in which one or more amino acid residues that are crucial for the biological activity of the encoded AHAS protein, are substituted for another amino acid residue or deleted).

It is understood that a “non-functional AHAS protein”, as used herein, refers to an AHAS protein that is not able to participate in dimer and/or tetramer formation and/or does not influence the enzymatic activity of other wildtype or (missense) mutant AHAS proteins that may be present in the cell. A non-functional AHAS protein is encoded by a full knockout AHAS allele.

An active AHAS protein is encoded by an active AHAS allele and can be both a wildtype AHAS protein as well as a mutant AHAS protein that is still biological active but is not inhibited by AHAS-inhibiting herbicides (e.g. an AHAS protein encoded by a nucleic acid sequence comprising a missense mutation), i.e. a herbicide tolerant AHAS protein.

The term “mutant AHAS protein”, as used herein, refers to an AHAS protein encoded by a mutant AHAS nucleic acid sequence (“AHAS allele”) whereby the mutation results in a change in the amino acid sequence of the AHAS protein. A mutant AHAS may be a non-functional AHAS protein, whereby amino acids essential for biological activity have been substituted or deleted. Alternatively, a mutant AHAS protein can contain a mutation through which it becomes uninhibitable by AHAS-inhibiting herbicides. Preferable, such a herbicide tolerant or herbicide resistant AHAS protein, is still capable of performing its natural function, i.e. the synthesis of branched amino acids.

Examples of such mutant herbicide tolerant AHAS proteins are known in the art and are described for instance in Duggleby, et al., 2008; WO09/046,334, WO09/031,031, U.S. patent application Ser. No. 09/001,3424, which are all incorporated herein by reference. Mutant herbicide tolerant AHAS proteins comprising two or more amino acid substitutions are described for instance in WO08/124,495, which is also incorporated herein by reference.

TABLE 1
Overview of herbicide tolerant amino acid substitution is AHAS
proteins and their references, which are all incorporated herein
(all positions are standardized to the A. thaliana AHAS amino
acid sequence, i.e. corresponding to SEQ ID NO: 2)

| posi- | (substitution) | | |
|---|---|---|---|
| tion | species | reference | |
| 121 | (Gly → Ala) | Okuzaki et al., | Plant Mol Biol. 64(1-2), |
| | Rice | 2007 | p219-24. |
| | (Gly → Ala) | Shimizu et al., | Plant Physiol. 147(4), |
| | Tobacco | 2008 | p1976-83. |
| | (plastids) | | |
| 122 | (Ala → Val) | Chang and | Biochem J. 1; 333 (Pt 3), |
| | Arabidopsis | Duggleby | p765-77. |
| | | 1998 | |
| | (Ala → Thr) | Bernasconi et al., | J Biol Chem. 21; 270(29), |
| | Cocklebur | 1995 | p17381-5. |
| | (Ala → Val) | Shimizu et al., | Plant Physiol. 147(4), |
| | Tobacco | 2008 | p1976-83. |
| | (plastids) | | |
| 124 | (Met → Glu) | Ott et al., 1996 | J Mol Biol. 25; 263(2), |
| | Arabidopsis | | p359-68. |
| 155 | (Ala → Thr) | Bernasconi et al., | J Biol Chem. 21; 270(29), |
| | Maize | 1995 | p17381-5. |
| 197 | (Pro → Ser) | Haughn et al., | Mol Gen Genet 211: |
| | Arabidopsis | 1988 | 266-271 |
| | (Pro → Leu) | Sibony et al., | Weed Res 41, p509-522 |
| | Pigweed | 2001 | |
| | (Pro → His) | Yu et al., 2003 | Weed Science, 51(6)6, |
| | Wild Radish | | p831-838 |
| | (Pro → Thr) | Tal and Rubin | Resistant Pest Management |
| | Crown Daisy | 2004 | Newsletter. 13, p31-33. |
| | (Pro →Gln/Ala) | Lee et al., 1988 | EMBO J. 7(5), |
| | Tobacco | | p1241-1248. |
| | (Pro → Ser/Thr) | Ruiter et al., | Plant Mol Biol. 53(5), |
| | Canola | 2003 | p675-89. |
| | (Pro → Ser) | Shimizu et al., | Plant Physiol. 147(4): |
| | Tobacco | 2008 | 1976-83. |
| | (plastids) | | |
| 199 | (Arg → Glu) | Ott et al., 1996 | J Mol Biol. 25; 263(2), |
| | Arabidopsis | | p359-68. |
| 205 | (Ala → Val) | Kolkman et al., | Theor Appl Genet. 109(6), |
| | Sunflower | 2004 | p1147-59 |
| 256 | (Arg → Phe/Gln) | Yoon et al., 2002 | Biochem Biophys Res |
| | Tobacco | | Commun. 293(1), p433-9. |
| 351 | (Met→ Cys) | Le et al., 2003 | Biochem. and Biophys. |
| | Tobacco | | Res. Commun. 306(4), |
| | | | p1075-1082 |
| 352 | (His → Gln) | Oh et al., 2001 | Biochem Biophys Res |
| | Tobacco | | Commun. 282(5), |
| | | | p1237-43. |
| 375 | (Asp → Ala) | Le et al., 2005 | Biochim Biophys Acta. |
| | Tobacco | | 1749(1), p103-12. |
| 376 | (Asp → Arg/Glu) | Le et al., 2005 | Biochim Biophys Acta. |
| | Tobacco | | 1749(1), p103-12. |
| | (Asp → Glu) | Whaley et al., | Weed Sci. Soc. Am. Abstr. |
| | Pigweed | 2004 | no. 161 |
| 570 | (Met → Cys) | Le et al., 2003 | Biochem Biophys |
| | Tobacco | | Res Commun 306(4), |
| | | | p1075-1082 |
| 571 | (Val → Gln) | Jung et al., | Biochem J. 383(Pt 1): |
| | Tobacco | 2004 | p53-61. |
| 574 | (Trp → Leu/Ser) | Chang and | Biochem J. 333 (Pt 3): |
| | Arabidopsis | Duggleby | p765-77. |
| | | 1998 | |
| | (Trp → Leu) | Lee et al., 1988 | EMBO J. 7(5): |
| | Tobacco | | p1241-1248. |
| | (Trp → Leu) | Hattori et al., | Mol Gen Genet. 246(4), |
| | Oilseed Rape | 1995 | p419-25. |
| | (Trp → Leu) | Bernasconi et al., | J Biol Chem. 270(29), |
| | Cocklebur | 1995 | p17381-5. |
| | (Trp →Cys/Ser) | Falco et al., 1989 | Dev Ind Microbiol 30, |
| | Cotton | | p187-194 |
| | (Trp → Leu) | Christoffers et al., | Weed Science 54(2), |
| | Wild Mustard | 2006 | p191-197 |
| 578 | (Phe →Asp/Glu) | Jung et al., 2004 | Biochem J. 383(Pt 1), |
| | Tobacco | | p53-61. |
| 653 | (Ser → Asn) | Chang and | Biochem J. 333 (Pt 3), |
| | Arabidopsis | Duggleby | p765-77. |
| | | 1998 | |
| | (Ser → Thr) | Lee et al., 1999 | FEBS Lett. 452(3), |
| | Arabidopsis | | p341-5. |
| | (Ser → Phe) | Lee et al., 1999 | FEBS Lett. 452(3), |
| | Arabidopsis | | p341-5. |
| | (Ser → Thr) | Chong and Choi | Biochem Biophys Res |
| | Tobacco | 2000 | Commun. 279(2), p462-7. |
| 654 | (Gly → Glu) | Croughan et al., | Clearfield rice: It's not a |
| | Rice | 2003 | GMO. Louisiana Agric. |
| | | | 46(4), p24-26. |

As used herein, a “herbicide” is a chemical substance used to destroy or inhibit the growth of plants, especially weeds. An “AHAS-inhibiting herbicide” or an “ALS-inhibiting herbicide” is a herbicide that interferes with the activity of the AHAS enzyme. Preferably, such an AHAS-inhibiting herbicide is a sulfonylurea herbicide, an imidazolinone herbicide, a sulfonylaminocarbonyltriazolinone herbicide, a triazolopyrimidine herbicide, a pyrimidyl(oxy/thio)benzoate herbicide, or mixture thereof. Examples of AHAS-inhibiting herbicides include for instance amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, quinclorac, rimsulfuron, sulfentrazone, sulfometuron, sulfosulfuron, thiencarbazone-methyl, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac.

As used herein, “thiencarbazone-methyl” is a herbicide also known as methyl 4-[(4,5-dihydro-3-methoxy-4-methyl-5-oxo-1H-1,2,4-triazol-1-yl)carbonylsulfamoyl]-5-methylthiophene-3-carboxylate (IUPAC) or methyl 4-[[[(4,5-dihydro-3-methoxy-4-methyl-5-oxo-1H-1,2,4-triazol-1-yl)carbonyl]amino]sulfonyl]-5-methyl-3-thiophenecarboxylate (CAS).

As used herein, “an increased herbicide tolerance” or “an increased herbicide resistance” refers to an AHAS protein (e.g. a mutant AHAS protein) which is significantly less inhibited by AHAS-inhibiting herbicides than a corresponding wildtype AHAS protein, but it can also refer to a naturally occurring variant that displays increased tolerance compared to e.g. AHAS proteins of other species. It also refers to plants comprising (alleles encoding) such herbicide tolerant AHAS proteins, which are significantly less disturbed in their normal growth and development by herbicides when compared to plants not comprising (alleles encoding) such herbicide tolerant AHAS proteins but instead comprising (alleles encoding) herbicide intolerant AHAS proteins.

The herbicide tolerance of an AHAS protein can be measured by methods known in the art such as a complementation assay in e.g. E. coli (WO08/124,495) or an AHAS enzyme assay (Singh et al., Anal. Biochem. 171:173-179, 1988). Alternatively, the herbicide tolerance of a plant comprising AHAS proteins can be evaluated by culturing (e.g. hypocotyl) explants of those plants on a growth medium, e.g. callus inducing medium, comprising the herbicide and subsequently measuring the growth of the explants under various herbicide concentrations.

As used herein, the preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell or seed lacking herbicide tolerant AHAS alleles and proteins, but that said amount does not kill or inhibit as severely the growth of the herbicide-resistant plants, plant tissues, plant cells, and seeds of the present invention. Typically, the effective amount of a herbicide is an amount that is routinely used in agricultural production systems to kill weeds of interest. Such an amount is known to those of ordinary skill in the art.

“Mutagenesis”, as used herein, refers to the process in which plant cells (e.g., a plurality of Brassica seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more AHAS alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, Brassica plants are regenerated from the treated cells using known techniques. For instance, the resulting Brassica seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant AHAS alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc. Additional techniques to screen for the presence of specific mutant AHAS alleles are described in the Examples below.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

“Crop plant” refers to plant species cultivated as a crop, such as, but not limited to, Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16). The definition does not encompass weeds, such as Arabidopsis thaliana.

The term “weed”, as used herein, refers to undesired vegetation on e.g. a field, or to plants, other then the intentionally planted crop plants, which grow unwantedly between the crop plants and may inhibit growth and development of the crop plants.

A “variety” is used herein in conformity with the UPOV convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

As used herein, the term “non-naturally occurring” or “cultivated” when used in reference to a plant, means a plant with a genome that has been modified by man. A transgenic plant, for example, is a non-naturally occurring plant that contains an exogenous nucleic acid molecule, e.g., a chimeric gene comprising a transcribed region which when transcribed yields a biologically active RNA molecule capable of reducing the expression of an endogenous gene, such as a AHAS gene according to the invention, and, therefore, has been genetically modified by man. In addition, a plant that contains a mutation in an endogenous gene, for example, a mutation in an endogenous AHAS gene, (e.g. in a regulatory element or in the coding sequence) as a result of an exposure to a mutagenic agent is also considered a non-naturally plant, since it has been genetically modified by man. Furthermore, a plant of a particular species, such as Brassica napus, that contains a mutation in an endogenous gene, for example, in an endogenous AHAS gene, that in nature does not occur in that particular plant species, as a result of, for example, directed breeding processes, such as marker-assisted breeding and selection or introgression, with a plant of the same or another species, such as Brassica juncea or rapa, of that plant is also considered a non-naturally occurring plant. In contrast, a plant containing only spontaneous or naturally occurring mutations, i.e. a plant that has not been genetically modified by man, is not a “non-naturally occurring plant” as defined herein and, therefore, is not encompassed within the invention. One skilled in the art understands that, while a non-naturally occurring plant typically has a nucleotide sequence that is altered as compared to a naturally occurring plant, a non-naturally occurring plant also can be genetically modified by man without altering its nucleotide sequence, for example, by modifying its methylation pattern.

As used herein, “an agronomically suitable plant development” refers to a development of the plant, in particular an oilseed rape plant, which does not adversely affect its performance under normal agricultural practices, more specifically its establishment in the field, vigor, flowering time, height, maturation, lodging resistance, yield, disease resistance, resistance to pod shattering, etc. Thus, lines with significantly increased herbicide tolerance with agronomically suitable plant development have herbicide tolerance that has increased as compared to other plants while maintaining a similar establishment in the field, vigor, flowering time, height, maturation, lodging resistance, yield, disease resistance, resistance to pod shattering, etc.

As used herein, “the nucleotide sequence of SEQ ID NO: Z from position X to position Y” indicates the nucleotide sequence including both nucleotide endpoints.

The term “comprising” is to be interpreted as specifying the presence of the stated parts, steps or components, but does not exclude the presence of one or more additional parts, steps or components. A plant comprising a certain trait may thus comprise additional traits.

It is understood that when referring to a word in the singular (e.g. plant or root), the plural is also included herein (e.g. a plurality of plants, a plurality of roots). Thus, reference to an element by the indefinite article “a” or “an” does not exclude the possibility that more than one of the element is present, unless the context clearly requires that there be one and only one of the elements. The indefinite article “a” or “an” thus usually means “at least one”.

### DETAILED DESCRIPTION

Brassica napus (genome AACC, 2n=4x=38), which is an allotetraploid (amphidiploid) species containing essentially two diploid genomes (the A and the C genome) due to its origin from diploid ancestors, is described to comprise five AHAS loci genes in its genome. AHAS2, AHAS3 and AHAS4 originate from the A genome, whereas AHAS1 and AHAS5 originate from the C genome. AHAS1 and AHAS3 are the only genes that are constitutively expressed and encode the primary AHAS activities essential to growth and development in B. napus (Tan et al., 2005).

In a mutagenized population of a Brassica napus plants, plants could be indentified bearing mutations in their AHAS genomic DNA that resulted in amino acid substitution (missense mutation), i.e. P179S in both AHAS1 and AHAS3, and that resulted in the introduction of premature stop codons. The P197S appeared to confer some level of SU tolerance. Surprisingly however, when combining the P197S mutation in one AHAS gene with a stop codon mutation in the other gene (full knockout allele), herbicide tolerance increased when compared to the P197S mutation in one gene only. It was found that the higher the contribution of the missense herbicide tolerant AHAS allele, to the AHAS multimer, by increasingly replacing the wildtype alleles with a combination of full knockout AHAS alleles and herbicide tolerant AHAS alleles, the higher the level of herbicide tolerance of the plant.

Thus, in a first embodiment the invention provides a Brassica plant comprising a full knockout AHAS allele.

As used herein, a “full knockout AHAS allele”, refers to a nucleic acid sequence of an AHAS gene, which encodes no functional AHAS protein, i.e. an AHAS protein that does not participate in nor influence AHAS dimer formation, or no AHAS protein at all. In one embodiment, a full knockout AHAS allele refers to any mutation (missense, nonsense or frameshift mutation) in the AHAS coding sequence that result in a disruption or deletion of at least one of the two dimer interfaces (encoding aa 119-217 or 508-607 of SEQ ID NO: 2, or aa 104-202 or 493-592 of SEQ ID NO: 4 or aa 101-199 or 490-589 of SEQ ID NO: 6) is thought to result in a full knockout AHAS allele as the encoded protein will not be able to participate in dimer formation.

In a particular embodiment, a full knockout AHAS allele can comprise a nonsense mutation, which is a mutation in a AHAS allele whereby one or more translation stop codons are introduced into the coding DNA and the corresponding mRNA sequence of the corresponding wild type AHAS allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. In one embodiment, a mutant AHAS allele comprising a nonsense mutation is an AHAS allele wherein an in-frame stop codon is introduced in the AHAS codon sequence by a single nucleotide substitution, such as HETO112, HETO102, HETO10 and HETO104. In another embodiment, a full knockout AHAS allele is an AHAS allele comprising a nonsense mutation whereby an in-frame stop codon is introduced in the AHAS coding sequence by double nucleotide substitutions. In yet another embodiment, a full knockout AHAS is an AHAS allele comprising a nonsense mutation whereby an in-frame stop codon is introduced in the AHAS coding sequence by triple nucleotide substitutions. The truncated protein lacks the amino acids encoded by the coding DNA downstream (3′) of the mutation (i.e. the C-terminal part of the AHAS protein) and maintains the amino acids encoded by the coding DNA upstream (5′) of the mutation (i.e. the N-terminal part of the AHAS protein). Thus, a mutant AHAS allele comprising a nonsense mutation anywhere upstream of or including the nucleotides encoding the second dimer interface (encoding aa 508-607 of SEQ ID NO: 2, or aa 493-592 of SEQ ID NO: 4 or aa 490-589 of SEQ ID NO: 6), will result in a full knockout AHAS allele. Also, an AHAS allele encoding an AHAS protein in which the amino acid corresponding to M542 and H142 of the Tobacco AHAS protein have been altered, as well as an AHAS protein wherein the regions between aa 567-582 and the region C-terminal of aa 630 corresponding to the Tobacco AHAS protein, have been altered, are thought to be full knockout AHAS alleles.

The invention also provides plants further comprising in its genome at least one second mutant AHAS allele, wherein the second mutant AHAS allele encodes a herbicide tolerant AHAS protein. Examples of herbicide tolerant AHAS proteins are described elsewhere in the application and in e.g. Duggleby et al. (Plant Phys. Biochem. 46, p309-324, 2008), WO08/124,495 and WO09/031,031. The person skilled in that art can, by choosing a particular herbicide tolerant AHAS allele, determine the tolerance of the plant to a particular AHAS-inhibiting herbicide. For instance, the P197S substitution will confer tolerance to e.g. thiencarbazone-methyl, whereas for instance the Ser to Asn substitution at residue 653 will confer tolerance to imidazolinone (Sathasivan et al., Plant Physiol. 97(3):1044-1050, 1991).

The amino acid sequence of such herbicide tolerant AHAS proteins according to the invention, or variants thereof, are amino acid sequences having at least 75%, at least 80%, at least 85%, at least 90%, at least 95%, 98%, 99% or 100% sequence identity with SEQ ID NO: 2, SEQ ID NO: 4 or SEQ ID NO: 6. These amino acid sequences may also be referred to as being “essentially similar” or “essentially identical” to the AHAS sequences provided in the sequence listing.

It will be understood that the more wildtype (non-herbicide-tolerant) AHAS alleles will be replaced by a combination of knockout and herbicide tolerant AHAS alleles in a plant, the more the AHAS multimer will be comprised of herbicide tolerant AHAS proteins and the greater the herbicide tolerance of the plant will be.

Thus, in another embodiment, plants are provided comprising only herbicide tolerant and full knockout AHAS alleles and no more wildtype (non-herbicide-tolerant) AHAS alleles of the active AHAS genes. This embodiment also encompasses plants in which all (non-herbicide-tolerant) wildtype alleles have been replaced by full knockout AHAS alleles, but wherein a herbicide tolerant AHAS encoding transgene has been introduced.

As used herein, active AHAS genes, refers to AHAS genes that contribute to AHAS protein function. In B. napus for instance, as described elsewhere in the application, only the AHAS1 and AHAS3 gene of the total of five AHAS genes present in the B. napus genome, are active AHAS genes.

It is also an embodiment of the invention to provide plant cells containing the mutant AHAS alleles of the invention. Gametes, seeds, embryos, either zygotic or somatic, progeny or hybrids of plants comprising the mutant AHAS alleles of the present invention, which are produced by traditional breeding methods, are also included within the scope of the present invention.

The invention further provides Brassica seeds selected from the group consisting of:
e) Brassica seed comprising AHAS1-HETO112 having been deposited at the NCIMB Limited on Dec. 17, 2009, under accession number NCIMB 41690;
f) Brassica seed comprising AHAS3-HETO102 having been deposited at the NCIMB Limited on Dec. 17, 2009, under accession number NCIMB 41687;
g) Brassica seed comprising AHAS3-HETO103 having been deposited at the NCIMB Limited on Dec. 17, 2009, under accession number NCIMB 41688; or
h) Brassica seed comprising AHAS3-HETO104 having been deposited at the NCIMB Limited on Dec. 17, 2009, under accession number NCIMB 41689;
   Also provided are a Brassica plant, or a cell, part, seed or progeny thereof, obtained from the above described seeds.

The invention further provides nucleic acid sequences representing full knockout AHAS alleles. Nucleic acid sequences of wild type AHAS alleles are represented in the sequence listing, while the mutant AHAS sequences (missense and knockout) of these sequences, and of sequences essentially similar to these, are described herein below and in the Examples, with reference to the wild type AHAS sequences.

“AHAS nucleic acid sequences” or “AHAS variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 2, SEQ ID NO: 4 or SEQ ID NO: 6 or nucleic acid sequences having at least 80%, at least 85%, at least 90%, at least 95%, 96%, 97%, 98%, 99% or 100% sequence identity with SEQ ID NO: 1 SEQ ID NO: 3 or SEQ ID NO: 5. These nucleic acid sequences may also be referred to as being “essentially similar” or “essentially identical” to the AHAS sequences provided in the sequence listing.

Provided are full knockout mutant AHAS nucleic acid sequences (comprising one or more mutations which result in no or a significantly reduced amount of functional encoded AHAS protein being produced or in no AHAS protein being produced) of AHAS genes. Such mutant nucleic acid sequences (referred to as ahas sequences) can be generated and/or identified using various known methods, as described further below, and are provided both in endogenous form and in isolated form. In one embodiment full knockout mutant AHAS nucleic acid sequences from Brassicaceae, particularly from Brassica species, especially from Brassica napus, but also from other Brassica crop species are provided. For example, Brassica species comprising an A and/or a C genome may comprise different alleles of AHAS genes, which can be identified and combined in a single plant according to the invention. In addition, mutagenesis methods can be used to generate mutations in wild type AHAS alleles, thereby generating mutant AHAS alleles for use according to the invention. Because specific AHAS alleles are preferably combined in a plant by crossing and selection, in one embodiment the AHAS nucleic acid sequences are provided within a plant (i.e. endogenously), e.g. a Brassica plant, preferably a Brassica plant which can be crossed with Brassica napus or which can be used to make a “synthetic” Brassica napus plant. Hybridization between different Brassica species is described in the art, e.g., as referred to in Snowdon (2007, Chromosome research 15: 85-95). Interspecific hybridization can, for example, be used to transfer genes from, e.g., the C genome in B. napus (AACC) to the C genome in B. carinata (BBCC), or even from, e.g., the C genome in B. napus (AACC) to the B genome in B. juncea (AABB) (by the sporadic event of illegitimate recombination between their C and B genomes). “Resynthesized” or “synthetic” Brassica napus lines can be produced by crossing the original ancestors, B. oleracea (CC) and B. rapa (AA). Interspecific, and also intergeneric, incompatibility barriers can be successfully overcome in crosses between Brassica crop species and their relatives, e.g., by embryo rescue techniques or protoplast fusion (see e.g. Snowdon, above).

The nucleic acid molecules may, thus, comprise one or more mutations, such as: a missense mutation, nonsense mutation or “STOP codon mutation, an insertion or deletion mutation, a frameshift mutation and/or a splice site mutation, as is already described in detail above. Basically, any mutation which results in a protein comprising at least one amino acid insertion, deletion and/or substitution relative to the wild type protein that leads to the formation of a non-functional AHAS protein or no AHAS protein at al results in a full knockout AHAS allele. It is, however, understood that mutations in certain parts of the protein are more likely to result in a non-functional AHAS protein, such as mutations leading to truncated proteins, whereby significant portions of the functional amino acid residues or domains, such as one of the dimer interfaces, are deleted or substituted.

Thus in one embodiment, nucleic acid sequences comprising one or more of any of the types of mutations described above are provided. In another embodiment, ahas sequences comprising one or more stop codon (nonsense) mutations are provided. Any of the above mutant nucleic acid sequences are provided per se (in isolated form), as are plants and plant parts comprising such sequences endogenously. In Table 2 herein below the most preferred full knockout AHAS alleles are described.

Mutant AHAS alleles may be generated (for example induced by mutagenesis) and/or identified using a range of methods, which are conventional in the art, for example using PCR based methods to amplify part or all of the AHAS genomic or cDNA.

Following mutagenesis, plants are grown from the treated seeds, or regenerated from the treated cells using known techniques. For instance, mutagenized seeds may be planted in accordance with conventional growing procedures and following self-pollination seed is formed on the plants. Alternatively, doubled haploid plantlets may be extracted from treated microspore or pollen cells to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed which is formed as a result of such self-pollination in the present or a subsequent generation may be harvested and screened for the presence of mutant AHAS alleles, using techniques which are conventional in the art, for example polymerase chain reaction (PCR) based techniques (amplification of the AHAS alleles) or hybridization based techniques, e.g. Southern blot analysis, BAC library screening, and the like, and/or direct sequencing of AHAS alleles. To screen for the presence of point mutations (so called Single Nucleotide Polymorphisms or SNPs) in mutant AHAS alleles, SNP detection methods conventional in the art can be used, for example oligoligation-based techniques, single base extension-based techniques, such as pyrosequencing, or techniques based on differences in restriction sites, such as TILLING.

Alternatively, plants or plant parts comprising one or more mutant AHAS alleles can be generated and identified using other methods, such as the “Delete-a-gene™” method which uses PCR to screen for deletion mutants generated by fast neutron mutagenesis (reviewed by Li and Zhang, 2002, Funct Integr Genomics 2:254-258), by the TILLING (Targeting Induced Local Lesions IN Genomes) method which identifies EMS-induced point mutations using denaturing high-performance liquid chromatography (DHPLC) to detect base pair changes by heteroduplex analysis (McCallum et al., 2000, Nat Biotech 18:455, and McCallum et al. 2000, Plant Physiol. 123, 439-442), etc. As mentioned, TILLING uses high-throughput screening for mutations (e.g. using Cel 1 cleavage of mutant-wildtype DNA heteroduplexes and detection using a sequencing gel system). Thus, the use of TILLING to identify plants or plant parts comprising one or more mutant AHAS alleles and methods for generating and identifying such plants, plant organs, tissues and seeds is encompassed herein. Thus in one embodiment, the method according to the invention comprises the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant plants.

Instead of inducing mutations in AHAS alleles, natural (spontaneous) mutant alleles may be identified by methods known in the art. For example, ECOTILLING may be used (Henikoff et al. 2004, Plant Physiology 135(2):630-6) to screen a plurality of plants or plant parts for the presence of natural mutant AHAS alleles. As for the mutagenesis techniques above, preferably Brassica species are screened which comprise an A and/or a C genome, so that the identified AHAS allele can subsequently be introduced into other Brassica species, such as Brassica napus, by crossing (inter- or intraspecific crosses) and selection. In ECOTILLING natural polymorphisms in breeding lines or related species are screened for by the TILLING methodology described above, in which individual or pools of plants are used for PCR amplification of the AHAS target, heteroduplex formation and high-throughput analysis. This can be followed by selecting individual plants having a required mutation that can be used subsequently in a breeding program to incorporate the desired mutant allele.

The identified mutant alleles can then be sequenced and the sequence can be compared to the wild type allele to identify the mutation(s). Optionally, whether a mutant allele functions as a herbicide tolerant or full knockout AHAS mutant allele can be tested as indicated above. Using this approach a plurality of mutant AHAS alleles (and Brassica plants comprising one or more of these) can be identified. The desired mutant alleles can then be combined with the desired wild type alleles by crossing and selection methods as described further below. Finally, a single plant comprising the desired number of mutant AHAS and the desired number of wild type and or herbicide tolerant AHAS alleles is generated.

Mutant AHAS alleles or plants comprising mutant AHAS alleles can be indentified or detected by method known in the art, such as direct sequencing, PCR based assays or hybridization based assays. Alternatively, methods can also be developed using the specific mutant AHAS allele specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage, incorporated herein by reference), RT-PCR-based detection methods, such as Taqman, or other detection methods, such as SNPlex. Briefly, in the Invader™ technology, the target mutation sequence may e.g. be hybridized with a labeled first nucleic acid oligonucleotide comprising the nucleotide sequence of the mutation sequence or a sequence spanning the joining region between the 5′ flanking region and the mutation region and with a second nucleic acid oligonucleotide comprising the 3′ flanking sequence immediately downstream and adjacent to the mutation sequence, wherein the first and second oligonucleotide overlap by at least one nucleotide. The duplex or triplex structure that is produced by this hybridization allows selective probe cleavage with an enzyme (Cleavase®) leaving the target sequence intact. The cleaved labeled probe is subsequently detected, potentially via an intermediate step resulting in further signal amplification.

The present invention also relates to the combination of specific AHAS alleles in one plant, to the transfer of one or more specific mutant AHAS allele(s) from one plant to another plant, to the plants comprising one or more specific mutant AHAS allele(s), the progeny obtained from these plants and to plant cells, plant parts, and plant seeds derived from these plants.

Thus, in one embodiment of the invention a method for transferring at least one selected full knockout AHAS allele from one plant to another plant is provided comprising the steps of:
a. generating and/or identifying a first plant comprising the at least one full knockout AHAS allele, as described above, or generating the first plant, as described above (wherein the first plant is homozygous or heterozygous for the at least one full knockout AHAS alleles)
b. crossing the first plant comprising the at least one full knockout AHAS allele with a second plant not comprising the at least one full knockout alleles, collecting F1 seeds from the cross (wherein the seeds are heterozygous for a full knockout AHAS allele if the first plant was homozygous for that full knockout AHAS allele, and wherein half of the seeds are heterozygous and half of the seeds are azygous for, i.e. do not comprise, a mutant AHAS allele if the first plant was heterozygous for that full knockout AHAS allele), and, optionally, identifying F1 plants comprising one or more selected full knockout AHAS alleles, as described above,
c. backcrossing F1 plants comprising at least one selected full knockout AHAS alleles with the second plant not comprising the at least one selected mutant AHAS alleles for one or more generations (x), collecting BCx seeds from the crosses, and identifying in every generation BCx plants comprising the at least one selected mutant AHAS alleles, as described above,

In another embodiment of the invention a method for combining a full knockout AHAS allele as described above, with a herbicide tolerant AHAS allele in one plant is provided comprising the steps of:
a. generating and/or identifying at least one plant comprising at least one selected full knockout AHAS allele and at least one plant comprising at least one selected herbicide tolerant AHAS allele, as described above,
b. crossing the first plant comprising at least one selected full knockout AHAS allele with a second plant comprising at least one selected herbicide tolerant AHAS allele, collecting F1 seeds from the cross, and, optionally, identifying an F1 plant comprising at least one selected full knockout AHAS allele from the first plant with at least one selected herbicide tolerant AHAS allele from the second plant, as described above,
c. optionally, repeating step (b) until an F1 plant comprising all selected AHAS alleles is obtained,

In another embodiment, the invention provides a method for producing a plant, in particular a Brassica crop plant, such as a Brassica napus plant, comprising a full knockout AHAS allele, but which preferably maintains an agronomically suitable development, is provided comprising combining and/or transferring AHAS alleles according to the invention in or to one plant, as described above

In yet another embodiment of the invention, a method for making a plant, in particular a Brassica crop plant, such as a Brassica napus plant, which is tolerant to herbicides, but which preferably maintains an agronomically suitable development, is provided comprising combining and/or transferring AHAS alleles according to the invention in or to one plant, as described above.

Methods are also provided for controlling weeds in the vicinity of crop plants, comprising the steps of:
a) planting in a field the seeds produced by the plant comprising at least one full knockout AHAS allele and at least one herbicide tolerant AHAS allele;
b) applying an effective amount of AHAS-inhibiting herbicide to the weeds and to the crop plants in the field to control the weeds; and
c) optionally, further comprising prior to step a) the step of applying an effective amount of AHAS-inhibiting herbicide to the field.

The invention also relates to the use of a full knockout AHAS allele of the invention to obtain a herbicide tolerant plant, in particular a Brassica crop plant, such as a Brassica napus plant, to obtain a herbicide tolerant plant.

The invention further relates to the use of a plant, in particular a Brassica crop plant, such as a Brassica napus plant, to produce seed comprising one or more full knockout AHAS alleles or to produce a crop of oilseed rape, comprising one or more full knockout AHAS allele(s).

It will be clear to the skilled artisan that the methods and means described herein are believed to be suitable for all plant cells and plants, both dicotyledonous and monocotyledonous plant cells and plants including but not limited to cotton, Brassica vegetables, oilseed rape, wheat, corn or maize, barley, alfalfa, peanuts, sunflowers, rice, oats, sugarcane, soybean, turf grasses, barley, rye, sorghum, sugar cane, vegetables (including chicory, lettuce, tomato, zucchini, bell pepper, eggplant, cucumber, melon, onion, leek), tobacco, potato, sugarbeet, papaya, pineapple, mango, Arabidopsis thaliana, but also plants used in horticulture, floriculture or forestry (poplar, fir, eucalyptus etc.).

### SEQUENCES

SEQ ID NO: 1: Genomic DNA/coding sequence of the AHAS1 gene from Arabidopsis thaliana (GenBank AY042819.1).
SEQ ID NO: 2: Amino acid sequence of the AHAS protein from Arabidopsis thaliana.
SEQ ID NO: 3: Genomic DNA/coding sequence of the AHAS1 gene from Brassica napus
SEQ ID NO: 4: Amino acid sequence of the AHAS1 protein from Brassica napus
SEQ ID NO: 5: Genomic DNA/coding sequence of the AHAS3 gene from Brassica napus
SEQ ID NO: 6: Amino acid sequence of the AHAS3 protein from Brassica napus

### EXAMPLES

### Example 1

### Generation and Isolation of Mutant Brassica AHAS Alleles

Mutations in AHAS1 and AHAS3 genes identified in Example 1 were generated and identified as follows:

30,000 seeds from an elite spring oilseed rape breeding line (M0 seeds) were preimbibed for two hours on wet filter paper in deionized or distilled water. Half of the seeds were exposed to 0.8% EMS and half to 1% EMS (Sigma: M0880) and incubated for 4 hours.

The mutagenized seeds (M1 seeds) were rinsed 3 times and dried in a fume hood overnight. 30,000 M1 plants were grown in soil and selfed to generate M2 seeds. M2 seeds were harvested for each individual M1 plant.

Two times 4800 M2 plants, derived from different M1 plants, were grown and DNA samples were prepared from leaf samples of each individual M2 plant according to the CTAB method (Doyle and Doyle, 1987, Phytochemistry Bulletin 19:11-15).

The DNA samples were screened for the presence of point mutations in the AHAS1 and AHAS3 genes causing amino acid substitutions (missense mutations) or the introduction of STOP codons (potential full knockout mutations) in the protein-encoding regions of the AHAS genes, by direct sequencing by standard sequencing techniques (Agowa) and analyzing the sequences for the presence of the point mutations using the NovoSNP software (VIB Antwerp).

The following mutant AHAS alleles were thus identified:

TABLE 2
Mutations in AHAS genes:

| | B. napus | | | | | A. thaliana | |
|---|---|---|---|---|---|---|---|
| | nt | aa | wt | mut | mut | nt | aa |
| allele | position | position | codon | codon | type | position | position |
| AHAS1 | SEQ ID 3 | SEQ ID 4 | | | | SEQ ID 1 | SEQ ID 2 |
| missense | | | | | | | |
| HETO108 | 544 | 182 | CCT | TCT | Pro→Ser | 589 | 197 |
| knockout | | | | | | | |
| HETO112¹ | 826 | 276 | CAG | TAG | Gln→stop | 871 | 291^{¶} |
| AHAS3 | SEQ ID 5 | SEQ ID 6 | | | | SEQ ID 1 | SEQ ID 2 |
| missense | | | | | | | |
| HETO111 | 535 | 179 | CCT | TCT | Pro→Ser | 589 | 197 |
| knockout | | | | | | | |
| HETO102² | 808 | 270^{¶} | CAG^{#} | TAG^{#} | Gln→stop | 862 | 288^{¶} |
| HETO103³ | 721 | 241^{¶} | CAG* | TAG* | Gln→stop | 775 | 259^{¶} |
| HETO104⁴ | 746 | 249^{¶} | TGG | TAG | Trp→stop | 800 | 267^{¶} |

*A. thaliana: wt codon CAA, mut codon TAA
^{#}A. thaliana: wt codon CAT, mut codon TAT, mut type His→Tyr
^{¶}aa → stop
¹Seeds comprising HETO112 (designated 09MB BN001441) have been deposited at the NCIMB Limited (Ferguson Building, Craibstone Estate, Bucksburn, Aberdeen, Scotland, AB21 9YA, UK) on Dec. 17, 2009, under accession number NCIMB 41690. Of the seeds, 25% is heterozygous for the HETO112 mutation, which can be identified using methods as described elsewhere in this application.
²Seeds comprising HETO102 (designated 09MB BN001437) have been deposited at the NCIMB Limited (Ferguson Building, Craibstone Estate, Bucksburn, Aberdeen, Scotland, AB21 9YA, UK) on Dec. 17, 2009, under accession number NCIMB 41687. Of the seeds, 25% is heterozygous for the HETO102 mutation, which can be identified using methods as described elsewhere in this application.
³Seeds comprising HETO103 (designated 09MB BN 001438) have been deposited at the NCIMB Limited (Ferguson Building, Craibstone Estate, Bucksburn, Aberdeen, Scotland, AB21 9YA, UK) on Dec. 17, 2009, under accession number NCIMB 41688. Of the seeds, 25% is heterozygous for the HETO103 mutation, which can be identified using methods as described elsewhere in this application.
⁴Seeds comprising HETO104 (designated 09MB BN001439) have been deposited at the NCIMB Limited (Ferguson Building, Craibstone Estate, Bucksburn, Aberdeen, Scotland, AB21 9YA, UK) on Dec. 17, 2009, under accession number NCIMB 41689. Of the seeds, 25% is heterozygous for the HETO104 mutation, which can be identified using methods as described elsewhere in this application.

In conclusion, the above examples show how mutant AHAS alleles can be generated and isolated. Also, plant material comprising such mutant alleles can be used to combine selected mutant and/or knockout alleles in a plant, as described in the following examples.

### Example 2

### Identification of a Brassica Plant Comprising a Mutant Brassica AHAS Allele

Brassica plants comprising the mutations in the AHAS genes identified in Example 1 were identified as follows:

For each mutant AHAS allele identified in the DNA sample of an M2 plant, at least 48 M2 plants derived from the same M1 plant as the M2 plant comprising the AHAS mutation were grown and DNA samples were prepared from leaf samples of each individual M2 plant.

The DNA samples were screened for the presence of the identified AHAS point mutations as described above in Example 1.

Heterozygous and homozygous (as determined based on the electropherograms) M2 plants comprising the same mutation were selfed and backcrossed, and BC1 seeds were harvested.

### Example 3

### Evaluation of Full Knockout AHAS Alleles

To asses whether the stop codon mutations (HETO102, HETO103, HETO104, HETO112) indeed resulted in full knockout AHAS alleles, i.e. encoding an AHAS protein not able to dimerize or encoding no AHAS protein at all, the following crossings were performed:

### Single BC1 Cross:

(+=wildtype allele, −=mutant allele)

### AHAS1 +/−X AHAS3 +/−

Resulting in double BC1 plants:

### 25% AHAS1 +/−, AHAS3 +/+, 25% AHAS1 +/−, AHAS3 +/−, 25% AHAS1 +/+, AHAS3 +/− and 25% AHAS1 +/+, AHAS3 +/+.

### Double BC2 Cross:

AHAS1 +/−, AHAS3 +/− (selected double BC1 plant) X AHAS1 +/+, AHAS3 +/+
Expected to result in double BC2 plants:

### 25% AHAS1 +/−, AHAS3 +/−, 25% AHAS1 +/−, AHAS3 +/+, 25% AHAS1 +/+, AHAS3 +/− and 25% AHAS1 +/+, AHAS3 +/+

Of each AHAS1 +/−, AHAS3 +/− X AHAS1 +/+, AHAS3 +/+ crosses, 24 progeny plants (double BC2) were analyzed for genotype by direct sequencing (Table 3).

TABLE 3
Observed genotype distribution of AHAS
knockout alleles in double BC2 crosses

| | HETO112/ | HETO112/ | HETO112/ |
|---|---|---|---|
| | HETO102 | HETO103 | HETO104 |
| AHAS1 +/−, AHAS3 +/− | — | — | — |
| AHAS1 +/−, AHAS3 +/+ | 9 | 6 | 7 |
| AHAS1 +/+, AHAS3 +/− | 5 | 12 | 8 |
| AHAS1 +/+, AHAS3 +/+ | 10 | 6 | 9 |

(+ = wildtype allele, − = mutant allele)

Since no double heterozygous BC1 plants were recovered, these results indicate that pollen comprising both an AHAS1 and an AHAS3 knockout allele are non-viable, suggesting that the HETO102, HETO103, HETO104 and HETO112 stop codon mutations indeed function as full knockout alleles.

### Example 4

### Measurement of Herbicide Tolerance of Brassica Plants Comprising Mutant AHAS Alleles

The correlation between the presence of missense and/or full knockout AHAS alleles in a Brassica plant grown in the greenhouse and tolerance to thiencarbazone-methyl was determined as follows. Of the Brassica plants identified in Example 1 and 2, crosses were made to obtain plants comprising both mutant AHAS1 and AHAS3 alleles (F1), which were subsequently backcrossed and selfed (BC1S1). Single gene AHAS missense mutants were backcrossed twice (BC2). These BC1S1 (representing all possible genotype combinations), as well as BC2 single AHAS gene mutant plants (50%+/+, 50%+/−), were sown in a greenhouse. Treatment post-emergence at the 1-2 leaf stage was carried out with a dose of 5 g a.i./ha of thiencarbazone-methyl and surviving plants were transplanted to 9 cm pots 10 days after spraying. The plants were evaluated for phenotype (height, side branching and leave morphology) 20 days after transplantation on scale of 5 to 1, where; type 5=normal (corresponding to wildtype unsprayed phenotype); type 4=normal height, some side branching, normal leaves; type 3=intermediate height, intermediate side branching, normal leaves; type 2=short, severe side branching (“bushy”), some leave malformations; type 1=short, severe side branching (“bushy”), severe leave malformations (Table 4).

TABLE 4
Tolerance rating upon spay testing (5 g a.i./ha
thiencarbazone-methyl), indicating number of seeds
that were sown (sown), number of seeds that germinated
(germ), number of surviving plants that were transplanted
to 9 cm pots after spraying (trans) and number of surviving
plants in each phenotype category (plant type).

| | | | | Plant type | | | | |
|---|---|---|---|---|---|---|---|---|
| Allele combination | sown | germ | trans | 1 | 2 | 3 | 4 | 5 |
| BC1S1 | | | | | | | | |
| HETO108/HETO102 | 204 | 201 | 169 | 108 | 34 | 21 | 1 | 0 |
| HETO108/HETO103 | 204 | 199 | 151 | 88 | 18 | 41 | 4 | 0 |
| HETO108/HETO104 | 204 | 200 | 115 | 66 | 18 | 28 | 2 | 1 |
| HETO112/HETO111 | 204 | 204 | 152 | 94 | 23 | 35 | 0 | 0 |
| BC2 | | | | | | | | |
| HETO108 | 51 | 50 | 22 | 20 | 2 | 0 | 0 | 0 |
| HETO111 | 51 | 49 | 25 | 22 | 0 | 0 | 0 | 0 |
| wt | 51 | 51 | 0 | 0 | 0 | 0 | 0 | 0 |

Of the BC2 seeds comprising HETO108 or HETO111 alone, about half of the germinated seeds survived spraying, which all grew out into type 1 or type 2 plants. This indicates that the AHAS1-P197S and the AHAS3-P197S mutation both confer herbicide tolerance, and that most likely these surviving plant were plants heterozygous for a single missense mutation (AHAS1 HETO108/+, AHAS3 +/+ and AHAS1 +/+, AHAS3 HETO111/+), whereas the non-surviving plants were the wildtype segregants (AHAS1 +/+, AHAS3 +/+). Surprisingly, when combining the P197S mutation in one AHAS gene with a knock-out allele in the other AHAS gene (HETO108/HETO102, HETO108/HETO103, HETO108/HETO104, HETO112/HETO111) in BC1S1 plants, about ¾ of the germinated seeds survived spraying, of which about ¼ grew out into type 3 plants and the rest into type 2 or 1. This suggests that the ¼ of non-surviving plants were again the wildtype segregants and the surviving plants contain the mutant alleles.

Next, of two P197S-knockout combinations, HETO108/HETO104 and HETO111/HETO112, ten plants (if available) of each plant type were genotyped by direct sequencing (Table 5). When comparing the genotype distributions per plant type, there appeared to be a gradual increase in the amount of missense alleles as well as the amount of full knockout alleles from type 1 to type 3, 4 and 5. The ratio of missense alleles to active AHAS alleles (missense alleles+wildtype alleles) also increased with plant type from an average of 0.32 and 0.33 for type 1 plants, an average of 0.65 and 0.65 for type 2 plants to an average of 0.74 and 0.88 for type 3 plants, for HETO108/HETO104 and HETO111/HETO112 respectively. Type 4 and 5 plants were only observed in the HETO108/HETO104 plants, of which the type 4 plants displayed an average missense to active allele ratio of 0.83. The one type 5 plant was probably missed during spraying.

TABLE 5
genotype distribution per plant type (+ = wildtype allele, − = mutant allele)

| | Allele | | | Missense | Allele | | | Missense |
|---|---|---|---|---|---|---|---|---|
| | combination | | Mutant | alleles/ | combination | | Mutant | alleles/ |
| | AHAS1 | AHAS3 | gene | Active | AHAS3 | AHAS1 | gene | Active |
| Type | HETO108 | HETO104 | dosage | alleles | HETO111 | HETO112 | dosage | alleles |
| 1 | +/− | +/+ | 1 | 1/4 | +/− | +/+ | 1 | 1/4 |
| | +/− | +/− | 2 | 1/3 | +/− | +/− | 2 | 1/3 |
| | +/− | +/+ | 1 | 1/4 | +/− | +/+ | 1 | 1/4 |
| | +/− | +/− | 2 | 1/3 | +/− | +/+ | 1 | 1/4 |
| | +/− | +/+ | 1 | 1/4 | +/− | −/− | 3 | 1/2 |
| | +/− | +/− | 2 | 1/3 | +/− | +/+ | 1 | 1/4 |
| | +/− | −/− | 3 | 1/2 | +/− | −/− | 3 | 1/2 |
| | +/− | +/− | 2 | 1/3 | +/− | +/− | 2 | 1/3 |
| | +/− | +/+ | 1 | 1/4 | +/− | +/− | 2 | 1/3 |
| | +/− | +/− | 2 | 1/3 | +/− | +/+ | 1 | 1/4 |
| 2 | −/− | +/+ | 2 | 2/4 | −/− | +/− | 3 | 2/3 |
| | −/− | −/− | 4 | 2/2 | −/− | +/− | 3 | 2/3 |
| | failed | failed | | | −/− | +/+ | 2 | 2/4 |
| | −/− | +/+ | 2 | 2/4 | −/− | +/− | 3 | 2/3 |
| | −/− | +/− | 3 | 2/3 | −/− | +/− | 3 | 2/3 |
| | −/− | +/− | 3 | 2/3 | −/− | +/− | 3 | 2/3 |
| | +/− | +/− | 2 | 1/2 | −/− | +/− | 3 | 2/3 |
| | −/− | −/− | 4 | 2/2 | +/− | +/− | 2 | 1/3 |
| | −/− | +/+ | 2 | 2/4 | −/− | +/− | 3 | 2/3 |
| | +/− | −/− | 3 | 1/2 | −/− | −/− | 4 | 2/2 |
| 3 | −/− | +/− | 3 | 2/3 | −/− | +/− | 3 | 2/3 |
| | −/− | +/− | 3 | 2/3 | −/− | −/− | 4 | 2/2 |
| | −/− | +/− | 3 | 2/3 | −/− | −/− | 4 | 2/2 |
| | −/− | +/− | 3 | 2/3 | −/− | −/− | 4 | 2/2 |
| | −/− | −/− | 4 | 2/2 | failed | failed | | |
| | −/− | +/− | 3 | 2/3 | failed | failed | | |
| | failed | failed | — | — | −/− | −/− | 4 | 2/2 |
| | −/− | −/− | 4 | 2/2 | −/− | +/− | 3 | 2/3 |
| | −/− | +/− | 3 | 2/3 | −/− | +/− | 3 | 2/3 |
| | −/− | +/− | 3 | 2/3 | −/− | −/− | 4 | 2/2 |
| 4 | −/− | +/− | 3 | 2/3 | | | | |
| | −/− | −/− | 4 | 2/2 | | | | |
| 5 | −/− | +/+ | 2 | 2/4 | | | | |

These result indicate that the higher the contribution of the herbicide tolerant AHAS protein to the AHAS protein pool, the higher the level of herbicide tolerance of the plant.

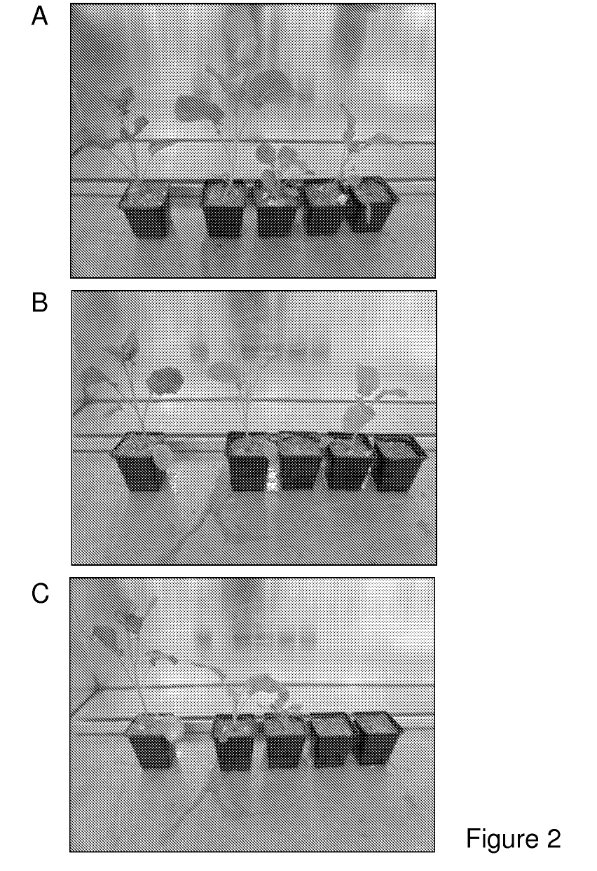

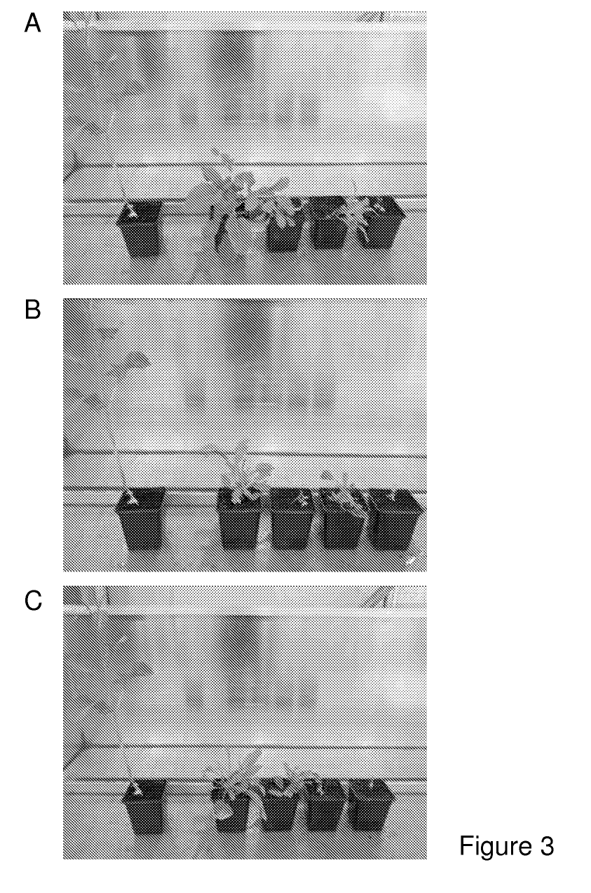

In another experiment, the effect of combining AHAS full knockouts with AHAS missense herbicide tolerant alleles on tolerance to thiencarbazone-methyl pre-planting application and thiencarbazone-methyl post-emergence spraying was tested in the greenhouse. To this end, the Brassica plants identified in Example 1 and 2 were backcrossed two times with an elite parent line, and subsequently selfed twice to obtain homozygous plants (BC2S2). Treatment pre-planting was carried out on the soil just after sowing with a dose of 20 g a.i./ha of thiencarbazone methyl. For assessment of vigor scores, plants were evaluated on a scale of 1 to 9, where 1=dead, 3=poor, 6=some aberrant phenotype and 9=vigorous. The vigor scores are an average of the scores taken at 2, 3 and 4 weeks after treatment. Treatment post-emergence at the first leaf stage was carried out with a dose of 10 g a.i./ha of thiencarbazone-methyl. The vigor scores are an average of the scores taken 1, 2 and 3 weeks after the treatment. The average values (Av) and standard deviations (SD) of the vigor scores are represented in Table 6. Representative pictures of the plants after treatment are shown in FIGS. 2 and 3.

TABLE 6
Average (Av) and standard deviation (SD) of vigor scores upon
spay testing pre-planting (pre) and post-emergence (post).

| Allele combination | | Pre | | Post | |
|---|---|---|---|---|---|
| AHAS1 | AHAS3 | Av | SD | Av | SD |
| HETO108/HETO108 | HETO111/HETO111 | 7.6 | 1.0 | 4.7 | 0.3 |
| HETO108/HETO108 | +/+ | 4.4 | 0.1 | 3.3 | 0.3 |
| +/+ | HETO111/HETO111 | 5.2 | 1.8 | 3.3 | 0 |
| +/+ | +/+ | 1.4 | 0.1 | 1.7 | 0 |
| HETO112/HETO112 | HETO111/HETO111 | 5.6 | 0.8 | 3.6 | 0.4 |
| HETO112/HETO112 | +/+ | 1.4 | 0.1 | 1.7 | 0 |
| +/+ | HETO111/HETO111 | 4.8 | 0.5 | 3.1 | 0.2 |
| +/+ | +/+ | 1.4 | 0.1 | 1.7 | 0 |
| HETO108/HETO108 | HETO104/HETO104 | 6.1 | 0.5 | 3.8 | 0.2 |
| HETO108/HETO108 | +/+ | 5.3 | 1.4 | 3.4 | 0.2 |
| +/+ | HETO104/HETO104 | 1.3 | 0 | 1.8 | 0.2 |
| +/+ | +/+ | 1.3 | 0 | 1.7 | 0 |

| | | | | |
|---|---|---|---|---|
| Elite parent line treated | 1.8 | 0 | 1.8 | 0.2 |
| Elite parent line untreated | 9 | 0 | 9 | 0 |

+ = wild-type allele.

Table 6 and FIGS. 2 and 3 show that, both upon pre-planting treatment and upon post-emergence spraying with thiencarbazone-methyl, plants in which one AHAS gene is homozygous for a missense herbicide tolerant allele and in which the other AHAS gene is homozygous for a full knock-out allele show a higher thiencarbazone-methyl tolerance than plants in which one AHAS gene is homozygous for a missense herbicide tolerant allele and the other AHAS gene is homozygous wild-type. These results further support the notion that the higher the contribution of the herbicide tolerant AHAS protein to the AHAS protein pool, the higher the level of herbicide tolerance of the plant.

### Example 5

### Measurement of Herbicide Tolerance of Brassica Plants Comprising Mutant AHAS Alleles in the Field

Tests were set up and conducted to asses the growth and performance of plants comprising AHAS full knock-out alleles, and to further analyze the correlation between the presence of full knockout and missense AHAS genes in Brassica plants and plant growth and herbicide tolerance of the Brassica plants in the field. To this end, the Brassica plants identified in Example 1 and 2 were backcrossed two times with an elite parent line, and subsequently selfed twice to obtain homozygous plants (BC2S2). Plants were grown as row plots in a split plot design with three replicates (main plots=herbicide treatments, subplots=genotypes) at two locations in Canada. Treatment pre-planting was carried out on the soil about two days before sowing with a dose of 0 (treatment A), 10 (treatment B), 20 (treatment C) or 30 (treatment D) g a.i./ha of thiencarbazone methyl. Herbicide tolerance was measured by scoring for different parameters. The parameter emergence (ERG) was scored at the cotyledon stage on a scale 1-9, where 1 means late emergence and 9 means early emergence. Establishment was scored 14 days after sowing (EST1) and 21 days after sowing (EST2). Scores were from 1 to 9, where 1 is the worst establishment (least plants that emerged), and 9 is the best establishment (most plants emerged). Phytotoxicity (PPTOX) was determined after establishment. Plants were evaluated on a scale of 1 to 9, where 1=completely yellowing, 5=50% of plant is yellow and 9=no yellowing. The vigor scores (see above) were determined at 1-2 leaf stage (VIG1), 7 days after VIG1 (VIG2) and 14 days after VIG1 (VIG3). The average values (Av) and standard deviations (SD) of the scores for the different parameters are represented in Table 7a-g.

TABLE 7a
Average (Av) and standard deviation (SD) of emergence (ERG)
scores upon treatment with 0 (treatment A), 10 (treatment B),
20 (treatment C) or 30 (treatment D) g a.i./ha of thiencarbazone
methyl pre-planting. + = wild-type allele. All plants are
homozygous for the respective AHAS1 and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 8.33 | 1.15 | 5.00 | 0.00 |
| WT | WT | B | 1.00 | 0.00 | 3.00 | 0.00 |
| WT | WT | C | 1.00 | 0.00 | 1.67 | 0.58 |
| WT | WT | D | 1.00 | 0.00 | 1.33 | 0.58 |
| WT | HETO104 | A | 7.67 | 0.58 | 5.00 | 0.00 |
| WT | HETO104 | B | 2.00 | 1.73 | 2.00 | 0.00 |
| WT | HETO104 | C | 1.00 | 0.00 | 1.33 | 0.58 |
| WT | HETO104 | D | 1.00 | 0.00 | 1.00 | 0.00 |
| HETO108 | WT | A | 7.67 | 1.15 | 5.00 | 0.00 |
| HETO108 | WT | B | 6.67 | 0.58 | 4.33 | 0.58 |
| HETO108 | WT | C | 5.00 | 1.00 | 4.33 | 1.15 |
| HETO108 | WT | D | 4.67 | 1.53 | 5.00 | 0.00 |
| HETO108 | HETO104 | A | 8.00 | 1.00 | 5.00 | 1.00 |
| HETO108 | HETO104 | B | 6.00 | 1.00 | 4.00 | 1.00 |
| HETO108 | HETO104 | C | 4.00 | 1.00 | 5.00 | 0.00 |
| HETO108 | HETO104 | D | 5.00 | 1.73 | 4.67 | 0.58 |
| WT | WT | A | 7.67 | 0.58 | 5.33 | 0.58 |
| WT | WT | B | 2.33 | 2.31 | 3.33 | 1.15 |
| WT | WT | C | 1.00 | 0.00 | 2.33 | 1.15 |
| WT | WT | D | 1.00 | 0.00 | 1.33 | 0.58 |
| WT | HETO111 | A | 8.00 | 0.00 | 4.33 | 0.58 |
| WT | HETO111 | B | 5.33 | 1.15 | 5.00 | 1.00 |
| WT | HETO111 | C | 4.67 | 0.58 | 4.00 | 1.00 |
| WT | HETO111 | D | 4.33 | 1.15 | 4.00 | 1.00 |
| HETO108 | WT | A | 8.67 | 0.58 | 5.67 | 0.58 |
| HETO108 | WT | B | 6.33 | 0.58 | 4.33 | 0.58 |
| HETO108 | WT | C | 5.33 | 1.15 | 5.00 | 0.00 |
| HETO108 | WT | D | 5.00 | 1.00 | 3.33 | 2.08 |
| HETO108 | HETO111 | A | 8.33 | 0.58 | 5.00 | 0.00 |
| HETO108 | HETO111 | B | 5.33 | 0.58 | 4.33 | 0.58 |
| HETO108 | HETO111 | C | 4.67 | 1.15 | 5.00 | 0.00 |
| HETO108 | HETO111 | D | 5.00 | 1.00 | 4.33 | 1.15 |
| WT | WT | A | 8.67 | 0.58 | 5.00 | 0.00 |
| WT | WT | B | 1.00 | 0.00 | 3.67 | 0.58 |
| WT | WT | C | 1.00 | 0.00 | 1.67 | 0.58 |
| WT | WT | D | 1.00 | 0.00 | 1.33 | 0.58 |
| WT | HETO111 | A | 8.67 | 0.58 | 5.00 | 0.00 |
| WT | HETO111 | B | 5.33 | 1.15 | 4.67 | 0.58 |
| WT | HETO111 | C | 4.33 | 0.58 | 4.67 | 0.58 |
| WT | HETO111 | D | 4.33 | 1.53 | 3.67 | 1.53 |
| HETO112 | WT | A | 8.33 | 0.58 | 5.33 | 0.58 |
| HETO112 | WT | B | 1.67 | 1.15 | 1.67 | 0.58 |
| HETO112 | WT | C | 1.00 | 0.00 | 1.00 | 0.00 |
| HETO112 | WT | D | 1.00 | 0.00 | 1.00 | 0.00 |
| HETO112 | HETO111 | A | 8.33 | 1.15 | 4.67 | 0.58 |
| HETO112 | HETO111 | B | 4.33 | 0.58 | 4.33 | 1.15 |
| HETO112 | HETO111 | C | 5.00 | 1.00 | 4.00 | 1.00 |
| HETO112 | HETO111 | D | 5.33 | 0.58 | 3.00 | 1.73 |

| | | | | | |
|---|---|---|---|---|---|
| Elite parent line | A | 9.00 | 0.00 | 6.00 | 0.00 |
| Elite parent line | B | 2.00 | 1.00 | 5.00 | 1.00 |
| Elite parent line | C | 2.33 | 0.58 | 4.33 | 0.58 |
| Elite parent line | D | 2.00 | 0.00 | 3.00 | 1.00 |

TABLE 7b
Average (Av) and standard deviation (SD) of establishment
(EST1) scores upon treatment with 0 (treatment A), 10
(treatment B), 20 (treatment C) or 30 (treatment D) g a.i./ha
of thiencarbazone methyl pre-planting. + = wild-type
allele. All plants are homozygous for the respective AHAS1
and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 8.33 | 1.15 | 6.00 | 0.00 |
| WT | WT | B | 1.00 | 0.00 | 4.33 | 0.58 |
| WT | WT | C | 1.00 | 0.00 | 3.67 | 2.52 |
| WT | WT | D | 1.00 | 0.00 | 1.33 | 0.58 |
| WT | HETO104 | A | 7.67 | 0.58 | 5.67 | 0.58 |
| WT | HETO104 | B | 2.33 | 2.31 | 3.33 | 0.58 |
| WT | HETO104 | C | 1.00 | 0.00 | 2.33 | 1.53 |
| WT | HETO104 | D | 1.00 | 0.00 | 1.00 | 0.00 |
| HETO108 | WT | A | 7.67 | 1.15 | 6.33 | 0.58 |
| HETO108 | WT | B | 6.67 | 0.58 | 4.67 | 2.31 |
| HETO108 | WT | C | 5.00 | 1.00 | 4.67 | 2.31 |
| HETO108 | WT | D | 4.67 | 1.53 | 5.33 | 1.15 |
| HETO108 | HETO104 | A | 8.00 | 1.00 | 6.33 | 0.58 |
| HETO108 | HETO104 | B | 6.33 | 0.58 | 5.00 | 1.73 |
| HETO108 | HETO104 | C | 4.00 | 1.00 | 5.00 | 1.00 |
| HETO108 | HETO104 | D | 5.00 | 1.73 | 5.33 | 0.58 |
| WT | WT | A | 7.67 | 0.58 | 5.67 | 0.58 |
| WT | WT | B | 2.67 | 2.89 | 4.33 | 0.58 |
| WT | WT | C | 1.00 | 0.00 | 4.00 | 2.65 |
| WT | WT | D | 1.00 | 0.00 | 1.00 | 0.00 |
| WT | HETO111 | A | 8.00 | 0.00 | 6.00 | 1.00 |
| WT | HETO111 | B | 5.00 | 0.00 | 6.00 | 0.00 |
| WT | HETO111 | C | 5.33 | 0.58 | 4.67 | 2.31 |
| WT | HETO111 | D | 4.33 | 1.15 | 4.33 | 2.08 |
| HETO108 | WT | A | 8.67 | 0.58 | 5.67 | 0.58 |
| HETO108 | WT | B | 6.33 | 0.58 | 5.33 | 1.53 |
| HETO108 | WT | C | 6.00 | 1.00 | 5.67 | 0.58 |
| HETO108 | WT | D | 5.33 | 0.58 | 3.67 | 2.08 |
| HETO108 | HETO111 | A | 8.00 | 0.00 | 6.67 | 0.58 |
| HETO108 | HETO111 | B | 6.00 | 0.00 | 6.00 | 0.00 |
| HETO108 | HETO111 | C | 5.33 | 0.58 | 5.67 | 0.58 |
| HETO108 | HETO111 | D | 5.33 | 1.15 | 3.67 | 2.08 |
| WT | WT | A | 8.67 | 0.58 | 5.67 | 0.58 |
| WT | WT | B | 1.00 | 0.00 | 5.00 | 1.00 |
| WT | WT | C | 1.00 | 0.00 | 3.33 | 1.53 |
| WT | WT | D | 1.00 | 0.00 | 1.67 | 1.15 |
| WT | HETO111 | A | 8.67 | 0.58 | 6.00 | 0.00 |
| WT | HETO111 | B | 5.67 | 1.53 | 6.00 | 1.00 |
| WT | HETO111 | C | 4.67 | 1.15 | 4.67 | 1.53 |
| WT | HETO111 | D | 4.33 | 1.53 | 4.00 | 2.65 |
| HETO112 | WT | A | 8.33 | 0.58 | 6.67 | 0.58 |
| HETO112 | WT | B | 1.67 | 1.15 | 2.33 | 1.15 |
| HETO112 | WT | C | 1.00 | 0.00 | 1.33 | 0.58 |
| HETO112 | WT | D | 1.00 | 0.00 | 1.00 | 0.00 |
| HETO112 | HETO111 | A | 8.33 | 1.15 | 5.67 | 0.58 |
| HETO112 | HETO111 | B | 5.00 | 1.00 | 5.67 | 0.58 |
| HETO112 | HETO111 | C | 5.33 | 1.15 | 5.00 | 1.73 |
| HETO112 | HETO111 | D | 5.67 | 0.58 | 3.67 | 0.58 |

| | | | | | |
|---|---|---|---|---|---|
| Elite parent line | A | 9.00 | 0.00 | 7.67 | 0.58 |
| Elite parent line | B | 2.00 | 1.00 | 6.67 | 0.58 |
| Elite parent line | C | 2.33 | 0.58 | 4.67 | 2.31 |
| Elite parent line | D | 2.00 | 0.00 | 4.00 | 1.73 |

TABLE 7c
Average (Av) and standard deviation (SD) of establishment
(EST2) scores upon treatment with 0 (treatment A), 10
(treatment B), 20 (treatment C) or 30 (treatment D) g a.i./ha
of thiencarbazone methyl pre-planting. + = wild-type
allele. All plants are homozygous for the respective AHAS1
and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 8.33 | 1.15 | 6.33 | 0.58 |
| WT | WT | B | 1.00 | 0.00 | 4.33 | 0.58 |
| WT | WT | C | 1.00 | 0.00 | 3.67 | 2.52 |
| WT | WT | D | 1.00 | 0.00 | 1.33 | 0.58 |
| WT | HETO104 | A | 7.67 | 0.58 | 6.67 | 0.58 |
| WT | HETO104 | B | 2.33 | 2.31 | 3.33 | 0.58 |
| WT | HETO104 | C | 1.00 | 0.00 | 2.33 | 1.53 |
| WT | HETO104 | D | 1.00 | 0.00 | 1.00 | 0.00 |
| HETO108 | WT | A | 7.67 | 1.15 | 6.67 | 0.58 |
| HETO108 | WT | B | 6.67 | 0.58 | 5.00 | 2.65 |
| HETO108 | WT | C | 5.00 | 1.00 | 4.67 | 2.31 |
| HETO108 | WT | D | 5.67 | 1.53 | 5.33 | 1.15 |
| HETO108 | HETO104 | A | 7.67 | 1.15 | 7.00 | 1.00 |
| HETO108 | HETO104 | B | 6.33 | 0.58 | 5.00 | 1.73 |
| HETO108 | HETO104 | C | 4.33 | 0.58 | 5.00 | 1.00 |
| HETO108 | HETO104 | D | 5.33 | 1.53 | 5.33 | 0.58 |
| WT | WT | A | 7.67 | 0.58 | 6.00 | 0.00 |
| WT | WT | B | 2.00 | 1.73 | 4.33 | 0.58 |
| WT | WT | C | 1.00 | 0.00 | 4.00 | 2.65 |
| WT | WT | D | 1.00 | 0.00 | 1.33 | 0.58 |
| WT | HETO111 | A | 8.00 | 0.00 | 7.67 | 1.15 |
| WT | HETO111 | B | 5.33 | 0.58 | 6.33 | 0.58 |
| WT | HETO111 | C | 4.67 | 1.15 | 4.67 | 2.31 |
| WT | HETO111 | D | 5.00 | 1.00 | 4.33 | 2.08 |
| HETO108 | WT | A | 8.33 | 0.58 | 6.33 | 0.58 |
| HETO108 | WT | B | 6.33 | 0.58 | 5.33 | 1.53 |
| HETO108 | WT | C | 5.67 | 1.53 | 6.00 | 0.00 |
| HETO108 | WT | D | 5.33 | 0.58 | 4.00 | 2.00 |
| HETO108 | HETO111 | A | 8.33 | 0.58 | 7.33 | 0.58 |
| HETO108 | HETO111 | B | 6.33 | 0.58 | 6.00 | 0.00 |
| HETO108 | HETO111 | C | 5.67 | 1.15 | 5.33 | 1.15 |
| HETO108 | HETO111 | D | 6.00 | 1.00 | 4.00 | 2.00 |
| WT | WT | A | 8.67 | 0.58 | 6.67 | 0.58 |
| WT | WT | B | 1.00 | 0.00 | 5.00 | 1.00 |
| WT | WT | C | 1.00 | 0.00 | 3.00 | 2.00 |
| WT | WT | D | 1.00 | 0.00 | 2.00 | 1.73 |
| WT | HETO111 | A | 8.67 | 0.58 | 6.67 | 0.58 |
| WT | HETO111 | B | 5.67 | 1.53 | 5.33 | 0.58 |
| WT | HETO111 | C | 4.67 | 1.15 | 4.67 | 1.53 |
| WT | HETO111 | D | 5.00 | 1.73 | 4.00 | 2.65 |
| HETO112 | WT | A | 8.00 | 0.00 | 7.67 | 0.58 |
| HETO112 | WT | B | 1.67 | 1.15 | 2.67 | 0.58 |
| HETO112 | WT | C | 1.00 | 0.00 | 1.67 | 0.58 |
| HETO112 | WT | D | 1.00 | 0.00 | 1.00 | 0.00 |
| HETO112 | HETO111 | A | 8.33 | 1.15 | 6.67 | 0.58 |
| HETO112 | HETO111 | B | 5.67 | 1.15 | 5.67 | 0.58 |
| HETO112 | HETO111 | C | 5.33 | 1.15 | 5.00 | 1.73 |
| HETO112 | HETO111 | D | 5.67 | 0.58 | 3.67 | 0.58 |

| | | | | | |
|---|---|---|---|---|---|
| Elite parent line | A | 9.00 | 0.00 | 8.00 | 1.00 |
| Elite parent line | B | 2.00 | 1.00 | 6.00 | 0.00 |
| Elite parent line | C | 2.33 | 0.58 | 4.67 | 2.31 |
| Elite parent line | D | 2.00 | 0.00 | 4.00 | 1.73 |

TABLE 7d
Average (Av) and standard deviation (SD) of phytotoxicity
(PPTOX) scores upon treatment with 0 (treatment A), 10
(treatment B), 20 (treatment C) or 30 (treatment D) g a.i./ha
of thiencarbazone methyl pre-planting. + = wild-type
allele. All plants are homozygous for the respective AHAS1
and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 9.00 | 0.00 | 7.67 | 1.15 |
| WT | WT | B | 1.00 | 0.00 | 1.33 | 0.58 |
| WT | WT | C | 1.00 | 0.00 | 1.00 | 0.00 |
| WT | WT | D | 1.00 | 0.00 | 1.00 | 0.00 |
| WT | HETO104 | A | 9.00 | 0.00 | 7.00 | 2.00 |
| WT | HETO104 | B | 1.33 | 0.58 | 1.33 | 0.58 |
| WT | HETO104 | C | 1.00 | 0.00 | 1.00 | 0.00 |
| WT | HETO104 | D | 1.00 | 0.00 | 1.00 | 0.00 |
| HETO108 | WT | A | 8.67 | 0.58 | 8.33 | 1.15 |
| HETO108 | WT | B | 7.00 | 1.00 | 6.33 | 0.58 |
| HETO108 | WT | C | 5.00 | 1.00 | 5.33 | 2.08 |
| HETO108 | WT | D | 5.33 | 1.15 | 5.00 | 1.73 |
| HETO108 | HETO104 | A | 9.00 | 0.00 | 7.33 | 1.53 |
| HETO108 | HETO104 | B | 6.00 | 0.00 | 4.33 | 0.58 |
| HETO108 | HETO104 | C | 5.33 | 0.58 | 6.00 | 1.00 |
| HETO108 | HETO104 | D | 5.00 | 1.00 | 6.33 | 0.58 |
| WT | WT | A | 9.00 | 0.00 | 8.00 | 1.73 |
| WT | WT | B | 1.00 | 0.00 | 1.67 | 1.15 |
| WT | WT | C | 1.00 | 0.00 | 1.33 | 0.58 |
| WT | WT | D | 1.00 | 0.00 | 2.00 | 1.73 |
| WT | HETO111 | A | 9.00 | 0.00 | 7.33 | 1.53 |
| WT | HETO111 | B | 5.67 | 0.58 | 6.00 | 0.00 |
| WT | HETO111 | C | 5.00 | 1.00 | 5.67 | 0.58 |
| WT | HETO111 | D | 5.00 | 1.00 | 4.00 | 1.73 |
| HETO108 | WT | A | 9.00 | 0.00 | 9.00 | 0.00 |
| HETO108 | WT | B | 5.67 | 0.58 | 5.00 | 2.00 |
| HETO108 | WT | C | 5.67 | 0.58 | 7.00 | 1.00 |
| HETO108 | WT | D | 5.00 | 1.00 | 4.67 | 0.58 |
| HETO108 | HETO111 | A | 9.00 | 0.00 | 7.67 | 1.53 |
| HETO108 | HETO111 | B | 5.67 | 0.58 | 5.67 | 0.58 |
| HETO108 | HETO111 | C | 5.00 | 1.00 | 5.67 | 0.58 |
| HETO108 | HETO111 | D | 6.00 | 1.00 | 5.33 | 0.58 |
| WT | WT | A | 9.00 | 0.00 | 7.33 | 1.53 |
| WT | WT | B | 1.00 | 0.00 | 2.33 | 0.58 |
| WT | WT | C | 1.00 | 0.00 | 1.00 | 0.00 |
| WT | WT | D | 1.00 | 0.00 | 1.00 | 0.00 |
| WT | HETO111 | A | 9.00 | 0.00 | 7.67 | 1.53 |
| WT | HETO111 | B | 5.33 | 0.58 | 6.33 | 0.58 |
| WT | HETO111 | C | 5.00 | 0.00 | 5.33 | 2.89 |
| WT | HETO111 | D | 4.33 | 1.53 | 5.67 | 1.15 |
| HETO112 | WT | A | 9.00 | 0.00 | 7.67 | 1.15 |
| HETO112 | WT | B | 1.00 | 0.00 | 1.00 | 0.00 |
| HETO112 | WT | C | 1.00 | 0.00 | 1.00 | 0.00 |
| HETO112 | WT | D | 1.00 | 0.00 | 1.00 | 0.00 |
| HETO112 | HETO111 | A | 9.00 | 0.00 | 7.67 | 1.15 |
| HETO112 | HETO111 | B | 5.67 | 0.58 | 5.33 | 1.53 |
| HETO112 | HETO111 | C | 5.67 | 0.58 | 4.67 | 1.53 |
| HETO112 | HETO111 | D | 5.33 | 0.58 | 4.33 | 1.15 |

| | | | | | |
|---|---|---|---|---|---|
| Elite parent line | A | 9.00 | 0.00 | 8.33 | 1.15 |
| Elite parent line | B | 1.00 | 0.00 | 3.67 | 0.58 |
| Elite parent line | C | 1.00 | 0.00 | 1.67 | 0.58 |
| Elite parent line | D | 1.00 | 0.00 | 1.00 | 0.00 |

TABLE 7e
Average (Av) and standard deviation (SD) of vigor1 (VIG1)
scores upon treatment with 0 (treatment A), 10 (treatment B),
20 (treatment C) or 30 (treatment D) g a.i./ha of thiencarbazone
methyl pre-planting. + = wild-type allele. All plants are
homozygous for the respective AHAS1 and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 8.00 | 1.00 | 6.33 | 0.58 |
| WT | WT | B | 1.00 | 0.00 | 2.33 | 0.58 |
| WT | WT | C | 1.00 | 0.00 | 1.33 | 0.58 |
| WT | WT | D | 1.00 | 0.00 | 1.33 | 0.58 |
| WT | HETO104 | A | 8.33 | 0.58 | 6.00 | 1.00 |
| WT | HETO104 | B | 1.33 | 0.58 | 1.67 | 0.58 |
| WT | HETO104 | C | 1.00 | 0.00 | 1.33 | 0.58 |
| WT | HETO104 | D | 1.00 | 0.00 | 1.00 | 0.00 |
| HETO108 | WT | A | 7.67 | 1.53 | 7.33 | 0.58 |
| HETO108 | WT | B | 7.00 | 0.00 | 6.00 | 1.00 |
| HETO108 | WT | C | 5.67 | 1.15 | 5.00 | 2.65 |
| HETO108 | WT | D | 5.67 | 1.53 | 4.67 | 1.53 |
| HETO108 | HETO104 | A | 7.67 | 1.15 | 6.00 | 1.00 |
| HETO108 | HETO104 | B | 6.33 | 0.58 | 4.67 | 1.53 |
| HETO108 | HETO104 | C | 5.00 | 1.00 | 5.00 | 1.00 |
| HETO108 | HETO104 | D | 5.33 | 1.53 | 5.33 | 0.58 |
| WT | WT | A | 7.33 | 1.15 | 6.00 | 1.00 |
| WT | WT | B | 1.33 | 0.58 | 2.33 | 0.58 |
| WT | WT | C | 1.00 | 0.00 | 1.67 | 0.58 |
| WT | WT | D | 1.00 | 0.00 | 1.33 | 0.58 |
| WT | HETO111 | A | 8.00 | 0.00 | 6.67 | 0.58 |
| WT | HETO111 | B | 5.67 | 0.58 | 6.33 | 0.58 |
| WT | HETO111 | C | 5.00 | 1.00 | 5.33 | 1.15 |
| WT | HETO111 | D | 5.00 | 1.00 | 3.67 | 1.53 |
| HETO108 | WT | A | 8.67 | 0.58 | 7.00 | 1.00 |
| HETO108 | WT | B | 6.00 | 1.00 | 4.33 | 2.52 |
| HETO108 | WT | C | 6.00 | 1.00 | 6.67 | 0.58 |
| HETO108 | WT | D | 5.33 | 1.15 | 5.00 | 1.00 |
| HETO108 | HETO111 | A | 8.00 | 0.00 | 7.33 | 0.58 |
| HETO108 | HETO111 | B | 6.33 | 0.58 | 5.33 | 0.58 |
| HETO108 | HETO111 | C | 5.33 | 1.53 | 5.00 | 1.00 |
| HETO108 | HETO111 | D | 6.00 | 1.00 | 4.67 | 0.58 |
| WT | WT | A | 8.33 | 0.58 | 5.67 | 1.53 |
| WT | WT | B | 1.00 | 0.00 | 3.00 | 0.00 |
| WT | WT | C | 1.00 | 0.00 | 1.67 | 0.58 |
| WT | WT | D | 1.00 | 0.00 | 1.33 | 0.58 |
| WT | HETO111 | A | 8.67 | 0.58 | 6.33 | 0.58 |
| WT | HETO111 | B | 6.00 | 1.00 | 6.00 | 1.00 |
| WT | HETO111 | C | 5.33 | 0.58 | 5.00 | 2.65 |
| WT | HETO111 | D | 5.00 | 1.73 | 5.33 | 0.58 |
| HETO112 | WT | A | 7.33 | 1.15 | 6.33 | 0.58 |
| HETO112 | WT | B | 1.00 | 0.00 | 1.33 | 0.58 |
| HETO112 | WT | C | 1.00 | 0.00 | 1.00 | 0.00 |
| HETO112 | WT | D | 1.00 | 0.00 | 1.00 | 0.00 |
| HETO112 | HETO111 | A | 8.33 | 1.15 | 6.33 | 0.58 |
| HETO112 | HETO111 | B | 6.33 | 0.58 | 5.33 | 0.58 |
| HETO112 | HETO111 | C | 5.67 | 0.58 | 4.67 | 1.53 |
| HETO112 | HETO111 | D | 5.67 | 1.15 | 4.00 | 1.00 |

| | | | | | |
|---|---|---|---|---|---|
| Elite parent line | A | 9.00 | 0.00 | 7.67 | 1.53 |
| Elite parent line | B | 1.00 | 0.00 | 4.00 | 0.00 |
| Elite parent line | C | 1.33 | 0.58 | 2.00 | 1.00 |
| Elite parent line | D | 1.00 | 0.00 | 1.33 | 0.58 |

TABLE 7f
Average (Av) and standard deviation (SD) of vigor2 (VIG2)
scores upon treatment with 0 (treatment A), 10 (treatment B),
20 (treatment C) or 30 (treatment D) g a.i./ha of thiencarbazone
methyl pre-planting. + = wild-type allele. All plants are
homozygous for the respective AHAS1 and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 9.00 | 0.00 | 7.33 | 0.58 |
| WT | WT | B | 1.00 | 0.00 | 1.67 | 1.15 |
| WT | WT | C | 1.00 | 0.00 | 1.33 | 0.58 |
| WT | WT | D | 1.00 | 0.00 | 1.33 | 0.58 |
| WT | HETO104 | A | 9.00 | 0.00 | 6.33 | 1.15 |
| WT | HETO104 | B | 1.00 | 0.00 | 1.33 | 0.58 |
| WT | HETO104 | C | 1.00 | 0.00 | 1.00 | 0.00 |
| WT | HETO104 | D | 1.00 | 0.00 | 1.00 | 0.00 |
| HETO108 | WT | A | 9.00 | 0.00 | 7.67 | 0.58 |
| HETO108 | WT | B | 8.33 | 0.58 | 5.33 | 2.89 |
| HETO108 | WT | C | 6.67 | 0.58 | 6.33 | 2.08 |
| HETO108 | WT | D | 6.33 | 1.15 | 5.00 | 1.73 |
| HETO108 | HETO104 | A | 9.00 | 0.00 | 6.67 | 1.15 |
| HETO108 | HETO104 | B | 7.33 | 0.58 | 5.00 | 2.00 |
| HETO108 | HETO104 | C | 6.00 | 1.00 | 6.33 | 1.53 |
| HETO108 | HETO104 | D | 5.67 | 1.53 | 6.00 | 1.00 |
| WT | WT | A | 9.00 | 0.00 | 7.67 | 1.53 |
| WT | WT | B | 1.00 | 0.00 | 1.67 | 1.15 |
| WT | WT | C | 1.00 | 0.00 | 1.67 | 0.58 |
| WT | WT | D | 1.00 | 0.00 | 1.33 | 0.58 |
| WT | HETO111 | A | 9.00 | 0.00 | 7.33 | 0.58 |
| WT | HETO111 | B | 7.00 | 1.00 | 7.00 | 0.00 |
| WT | HETO111 | C | 6.33 | 0.58 | 6.00 | 2.65 |
| WT | HETO111 | D | 5.67 | 1.15 | 4.33 | 2.08 |
| HETO108 | WT | A | 9.00 | 0.00 | 7.67 | 0.58 |
| HETO108 | WT | B | 7.67 | 1.53 | 5.67 | 2.08 |
| HETO108 | WT | C | 6.67 | 1.15 | 7.67 | 0.58 |
| HETO108 | WT | D | 6.33 | 1.15 | 5.33 | 1.53 |
| HETO108 | HETO111 | A | 9.00 | 0.00 | 7.67 | 1.15 |
| HETO108 | HETO111 | B | 7.33 | 0.58 | 7.00 | 0.00 |
| HETO108 | HETO111 | C | 6.33 | 1.53 | 6.33 | 0.58 |
| HETO108 | HETO111 | D | 7.00 | 1.00 | 4.33 | 2.08 |
| WT | WT | A | 9.00 | 0.00 | 6.33 | 1.15 |
| WT | WT | B | 1.00 | 0.00 | 2.00 | 0.00 |
| WT | WT | C | 1.00 | 0.00 | 1.33 | 0.58 |
| WT | WT | D | 1.00 | 0.00 | 1.00 | 0.00 |
| WT | HETO111 | A | 9.00 | 0.00 | 7.33 | 0.58 |
| WT | HETO111 | B | 7.00 | 1.00 | 7.00 | 0.00 |
| WT | HETO111 | C | 6.33 | 0.58 | 5.67 | 3.21 |
| WT | HETO111 | D | 5.33 | 1.53 | 5.00 | 2.65 |
| HETO112 | WT | A | 8.67 | 0.58 | 7.00 | 0.00 |
| HETO112 | WT | B | 1.00 | 0.00 | 1.00 | 0.00 |
| HETO112 | WT | C | 1.00 | 0.00 | 1.00 | 0.00 |
| HETO112 | WT | D | 1.00 | 0.00 | 1.00 | 0.00 |
| HETO112 | HETO111 | A | 9.00 | 0.00 | 7.33 | 0.58 |
| HETO112 | HETO111 | B | 7.33 | 0.58 | 6.67 | 0.58 |
| HETO112 | HETO111 | C | 6.33 | 1.15 | 5.33 | 2.08 |
| HETO112 | HETO111 | D | 6.67 | 1.15 | 5.33 | 0.58 |

| | | | | | |
|---|---|---|---|---|---|
| Elite parent line | A | 9.00 | 0.00 | 8.00 | 1.00 |
| Elite parent line | B | 1.00 | 0.00 | 4.00 | 1.00 |
| Elite parent line | C | 1.00 | 0.00 | 2.00 | 1.00 |
| Elite parent line | D | 1.00 | 0.00 | 1.00 | 0.00 |

TABLE 7g
Average (Av) and standard deviation (SD) of vigor3 (VIG3)
scores upon treatment with 0 (treatment A), 10 (treatment B),
20 (treatment C) or 30 (treatment D) g a.i./ha of thiencarbazone
methyl pre-planting. + = wild-type allele. All plants are
homozygous for the respective AHAS1 and AHAS3 alleles.

| Allele combination | | | Location A | | Location B | |
|---|---|---|---|---|---|---|
| AHAS1 | AHAS3 | Treatment | Av | SD | Av | SD |
| WT | WT | A | 9.00 | 0.00 | 7.67 | 1.15 |
| WT | WT | B | 1.00 | 0.00 | 1.33 | 0.58 |
| WT | WT | C | 1.00 | 0.00 | 1.00 | 0.00 |
| WT | WT | D | 1.00 | 0.00 | 1.00 | 0.00 |
| WT | HETO104 | A | 9.00 | 0.00 | 6.67 | 1.53 |
| WT | HETO104 | B | 1.00 | 0.00 | 1.00 | 0.00 |
| WT | HETO104 | C | 1.00 | 0.00 | 1.00 | 0.00 |
| WT | HETO104 | D | 1.00 | 0.00 | 1.00 | 0.00 |
| HETO108 | WT | A | 9.00 | 0.00 | 8.33 | 1.15 |
| HETO108 | WT | B | 8.33 | 0.58 | 5.33 | 2.89 |
| HETO108 | WT | C | 7.33 | 1.15 | 6.33 | 2.08 |
| HETO108 | WT | D | 7.00 | 1.00 | 5.33 | 2.08 |
| HETO108 | HETO104 | A | 9.00 | 0.00 | 7.00 | 1.00 |
| HETO108 | HETO104 | B | 8.00 | 0.00 | 5.33 | 2.52 |
| HETO108 | HETO104 | C | 6.67 | 1.15 | 6.67 | 1.53 |
| HETO108 | HETO104 | D | 6.67 | 1.53 | 6.00 | 1.00 |
| WT | WT | A | 9.00 | 0.00 | 8.00 | 1.73 |
| WT | WT | B | 1.00 | 0.00 | 1.33 | 0.58 |
| WT | WT | C | 1.00 | 0.00 | 1.00 | 0.00 |
| WT | WT | D | 1.00 | 0.00 | 1.00 | 0.00 |
| WT | HETO111 | A | 9.00 | 0.00 | 8.00 | 1.00 |
| WT | HETO111 | B | 7.67 | 1.53 | 7.33 | 0.58 |
| WT | HETO111 | C | 6.67 | 0.58 | 6.00 | 2.65 |
| WT | HETO111 | D | 6.67 | 1.53 | 4.67 | 2.31 |
| HETO108 | WT | A | 9.00 | 0.00 | 8.00 | 0.00 |
| HETO108 | WT | B | 7.67 | 1.53 | 6.00 | 2.00 |
| HETO108 | WT | C | 7.00 | 1.00 | 8.00 | 1.00 |
| HETO108 | WT | D | 7.33 | 0.58 | 5.33 | 1.53 |
| HETO108 | HETO111 | A | 9.00 | 0.00 | 8.00 | 1.00 |
| HETO108 | HETO111 | B | 8.33 | 0.58 | 7.67 | 0.58 |
| HETO108 | HETO111 | C | 7.00 | 1.00 | 6.67 | 0.58 |
| HETO108 | HETO111 | D | 8.00 | 1.00 | 4.33 | 2.52 |
| WT | WT | A | 9.00 | 0.00 | 7.33 | 0.58 |
| WT | WT | B | 1.00 | 0.00 | 1.33 | 0.58 |
| WT | WT | C | 1.00 | 0.00 | 1.00 | 0.00 |
| WT | WT | D | 1.00 | 0.00 | 1.00 | 0.00 |
| WT | HETO111 | A | 9.00 | 0.00 | 8.33 | 0.58 |
| WT | HETO111 | B | 7.00 | 1.00 | 7.33 | 0.58 |
| WT | HETO111 | C | 6.67 | 0.58 | 6.00 | 3.61 |
| WT | HETO111 | D | 6.33 | 1.53 | 5.33 | 2.89 |
| HETO112 | WT | A | 9.00 | 0.00 | 7.00 | 0.00 |
| HETO112 | WT | B | 1.00 | 0.00 | 1.00 | 0.00 |
| HETO112 | WT | C | 1.00 | 0.00 | 1.00 | 0.00 |
| HETO112 | WT | D | 1.00 | 0.00 | 1.00 | 0.00 |
| HETO112 | HETO111 | A | 9.00 | 0.00 | 8.00 | 1.00 |
| HETO112 | HETO111 | B | 8.00 | 1.00 | 7.33 | 1.15 |
| HETO112 | HETO111 | C | 7.00 | 1.00 | 5.67 | 2.31 |
| HETO112 | HETO111 | D | 7.67 | 0.58 | 5.67 | 1.15 |

| | | | | | |
|---|---|---|---|---|---|
| Elite parent line | A | 9.00 | 0.00 | 8.33 | 1.15 |
| Elite parent line | B | 1.00 | 0.00 | 3.67 | 1.53 |
| Elite parent line | C | 1.00 | 0.00 | 1.00 | 0.00 |
| Elite parent line | D | 1.00 | 0.00 | 1.00 | 0.00 |

Table 7 shows that the presense of either knock-out allele in homozygous form surprisingly does not have a negative effect on overall plant appearance and growth in the field under non-treated conditions. Further, the contribution of the knock-out allele to herbicide tolerance conferred by the missense allele was calculated. First, the scores were corrected for a possible effect of the growth per se, independent of herbicide treatment. To this end, the scores for treatments B, C and D were divided by the scores for treatment A for the same genotype and for the same parameter (corrected herbicide tolerance scores). Next, the effect of the knock-out allele to these corrected herbicide tolerance scores obtained by the missense allele was calculated. Therefore, the corrected herbicide tolerance scores for the missense—knock-out allele combination was divided by the corrected herbicide tolerance scores for the missense allele—wild-type combination. In case the knock-out allele has no effect on herbicide tolerance conferred by the missense allele, this ratio should be 1. In case the knock-out allele positively contributes to the herbicide tolerance conferred by the missense allele, this ratio should be higher than 1. The results for the contribution of the knock-out allele to herbicide tolerance conferred by the missense allele as calculated above are shown in table 8.

TABLE 8
Relative contribution of the knock-out allele (HETO112 and
HETO104) to herbicide tolerance conferred by the missense
allele (HETO108 and HETO111). The relative effect is shown
on emergence (ERG), establishment 14 days after sowing
(EST1) and 21 days after sowing (EST2), phytotoxicity (PPTOX),
and vigor at 1-2 leaf stage (VIG1), 7 days after VIG1 (VIG2) and
14 days after VIG1 (VIG3).

| | | | | | | | |
|---|---|---|---|---|---|---|---|
| Treatment | ERG | EST1 | EST2 | PPTOX | VIG1 | VIG2 | VIG3 |

Contribution of HETO104 (AHAS3 KO) to herbicide tolerance
conferred by HETO108 (AHAS1 missense)
Location A

| | | | | | | | |
|---|---|---|---|---|---|---|---|
| B | 0.86 | 0.91 | 0.95 | 0.83 | 0.90 | 0.88 | 0.96 |
| C | 0.77 | 0.77 | 0.87 | 1.03 | 0.88 | 0.90 | 0.91 |
| D | 1.03 | 1.03 | 0.94 | 0.90 | 0.94 | 0.89 | 0.95 |

Location B

| | | | | | | | |
|---|---|---|---|---|---|---|---|
| B | 0.92 | 1.07 | 0.95 | 0.78 | 0.95 | 1.08 | 1.19 |
| C | 1.15 | 1.07 | 1.02 | 1.28 | 1.22 | 1.15 | 1.25 |
| D | 0.93 | 1.00 | 0.95 | 1.44 | 1.40 | 1.38 | 1.34 |

Contribution of HETO112 (AHAS1 KO) to herbicide tolerance
conferred by HETO111 (AHAS3 missense)
Location A

| | | | | | | | |
|---|---|---|---|---|---|---|---|
| B | 0.85 | 0.92 | 1.04 | 1.06 | 1.10 | 1.05 | 1.14 |
| C | 1.20 | 1.19 | 1.19 | 1.13 | 1.11 | 1.00 | 1.05 |
| D | 1.28 | 1.36 | 1.18 | 1.23 | 1.18 | 1.25 | 1.21 |

Location B

| | | | | | | | |
|---|---|---|---|---|---|---|---|
| B | 0.99 | 1.00 | 1.06 | 0.84 | 0.89 | 0.95 | 1.04 |
| C | 0.92 | 1.13 | 1.07 | 0.88 | 0.93 | 0.94 | 0.98 |
| D | 0.88 | 0.97 | 0.92 | 0.76 | 0.75 | 1.07 | 1.11 |

In table 8 it can be seen that there is, under certain conditions, a trend towards improved herbicide tolerance in the presence of the knock-out allele. For example, the knock-out allele HETO104 has a positive effect on PPTOX, VIG1, VIG2 and VIG3 at higher herbicide concentrations in location B, whereas the knock-out allele HETO112 has a positive effect on ERG, EST1, EST2, PPTOX, VIG1, VIG2 and VIG3 on medium to high herbicide concentrations in location A. The differences between locations A and B may be explained by the registered heavier rainfall after treatment in location B. This rainfall may also explain the slightly better performance of wild-type plants upon herbicide treatment in location B (table 7; rain may have diluted the herbicide concentration in the soil), as well as the slightly worse performance of wild-type plants without herbicide treatment in location B (table 7; suboptimal (wet) conditions for normal growth).

The correlation between the presence of full knockout and missense AHAS genes in Brassica plants on plant growth and herbicide tolerance of the Brassica plants in the field on location A was also tested upon post-emergence herbicide treatment. The field setup was the same as for the pre-planting field trial. Post-emergence treatment was carried out on the 2-4 leaf stage with a rate of 0 (treatment A), 10 (treatment B), 20 (treatment C) g a.i./ha of thiencarbazone methyl. The phytotoxicity (PPTOX) and vigor1 (VIG1; vigor scores 7 days after herbicide spray) were determined as described above. The average values (Av) and standard deviations (SD) of the scores for the different parameters are represented in Table 9.

TABLE 9
Average (Av) and standard deviation (SD) of phytotoxicity
(PPTOX) and vigor1 (VIG1) scores upon treatment with 0
(treatment A), 10 (treatment B), or 20 (treatment C) g a.i./ha of
thiencarbazone methyl post-emergence. + = wild-type
allele. All plants are homozygous for the respective AHAS1 and
AHAS3 alleles.

| Allele combination | | | PPTOX | | VIG1 | |
|---|---|---|---|---|---|---|
| AHAS1 | AHAS3 | Treatment | Av | SD | Av | SD |
| WT | WT | A | 9.00 | 0.00 | 9.00 | 0.00 |
| WT | WT | B | 1.00 | 0.00 | 1.67 | 0.58 |
| WT | WT | C | 1.00 | 0.00 | 2.00 | 0.00 |
| WT | HETO104 | A | 9.00 | 0.00 | 7.67 | 0.58 |
| WT | HETO104 | B | 1.00 | 0.00 | 1.67 | 0.58 |
| WT | HETO104 | C | 1.00 | 0.00 | 1.67 | 0.58 |
| HETO108 | WT | A | 9.00 | 0.00 | 9.00 | 0.00 |
| HETO108 | WT | B | 6.00 | 1.00 | 6.00 | 1.00 |
| HETO108 | WT | C | 4.33 | 0.58 | 4.67 | 0.58 |
| HETO108 | HETO104 | A | 9.00 | 0.00 | 9.00 | 0.00 |
| HETO108 | HETO104 | B | 5.00 | 1.00 | 5.00 | 1.00 |
| HETO108 | HETO104 | C | 4.00 | 0.00 | 4.00 | 0.00 |
| WT | WT | A | 9.00 | 0.00 | 9.00 | 0.00 |
| WT | WT | B | 1.00 | 0.00 | 1.67 | 0.58 |
| WT | WT | C | 1.00 | 0.00 | 2.00 | 0.00 |
| WT | HETO111 | A | 9.00 | 0.00 | 9.00 | 0.00 |
| WT | HETO111 | B | 5.33 | 0.58 | 5.33 | 0.58 |
| WT | HETO111 | C | 4.67 | 0.58 | 4.67 | 0.58 |
| HETO108 | WT | A | 9.00 | 0.00 | 9.00 | 0.00 |
| HETO108 | WT | B | 5.67 | 1.15 | 5.67 | 0.58 |
| HETO108 | WT | C | 4.33 | 0.58 | 4.33 | 0.58 |
| HETO108 | HETO111 | A | 9.00 | 0.00 | 9.00 | 0.00 |
| HETO108 | HETO111 | B | 6.67 | 0.58 | 6.67 | 0.58 |
| HETO108 | HETO111 | C | 6.00 | 1.00 | 6.00 | 0.00 |
| WT | WT | A | 9.00 | 0.00 | 9.00 | 0.00 |
| WT | WT | B | 1.00 | 0.00 | 1.67 | 0.58 |
| WT | WT | C | 1.00 | 0.00 | 2.00 | 0.00 |
| WT | HETO111 | A | 9.00 | 0.00 | 9.00 | 0.00 |
| WT | HETO111 | B | 5.67 | 0.58 | 5.33 | 0.58 |
| WT | HETO111 | C | 4.67 | 0.58 | 4.67 | 0.58 |
| HETO112 | WT | A | 9.00 | 0.00 | 8.67 | 0.58 |
| HETO112 | WT | B | 1.00 | 0.00 | 2.00 | 0.00 |
| HETO112 | WT | C | 1.00 | 0.00 | 1.67 | 0.58 |
| HETO112 | HETO111 | A | 9.00 | 0.00 | 9.00 | 0.00 |
| HETO112 | HETO111 | B | 5.00 | 0.00 | 5.00 | 0.00 |
| HETO112 | HETO111 | C | 4.67 | 0.58 | 4.67 | 0.58 |

| | | | | | |
|---|---|---|---|---|---|
| Elite parent line | A | 9.00 | 0.00 | 9.00 | 0.00 |
| Elite parent line | B | 1.00 | 0.00 | 2.33 | 0.58 |
| Elite parent line | C | 1.00 | 0.00 | 2.33 | 0.58 |

As shown in table 9, also in this field trial, there is no negative effect of the knock-out AHAS alleles on plant growth per se. With respect to the contribution of the knock-out alleles on herbicide tolerance upon post-emergence treatment, no conclusions can be drawn due to the limited number of data obtained from one location only.

In summary, the field results shown in tables 7, 8 and 9 show that, importantly, the presence of the knock-out alleles HETO112 (AHAS1) and HETO104 (AHAS3) in a homozygous state do not negatively affect plant growth in the field. Moreover, in table 8 it can be seen that under certain conditions, the knock-out AHAS alleles contribute positively to herbicide tolerance conferred by the missense alleles in the field.

### Example 6

### Detection and/or Transfer of Mutant Ahas Alleles into (Elite) Brassica Lines

The mutant AHAS genes are transferred into (elite) Brassica breeding lines by the following method: A plant containing a mutant AHAS gene (donor plant), is crossed with an (elite) Brassica line (elite parent/recurrent parent) or variety lacking the mutant AHAS gene. The following introgression scheme is used (the mutant AHAS allele is abbreviated to AHAS while the wild type is depicted as AHAS):

Initial cross: ahas/ahas (donor plant) X AHAS/AHAS (elite parent)
F1 plant: AHAS/ahas
BC1 cross: AHAS/ahas X AHAS/AHAS (recurrent parent)
BC1 plants: 50% AHAS/ahas and 50% AHAS/AHAS
The 50% ahas/AHAS are selected by direct sequencing or using molecular markers (e.g. AFLP, PCR, Invader™, TaqMan® and the like) for the mutant AHAS allele (ahas).
BC2 cross: AHAS/AHAS (BC1 plant) X AHAS/AHAS (recurrent parent)
BC2 plants: 50% AHAS/ahas and 50% AHAS/AHAS
The 50% AHAS/AHAS are selected by direct sequencing or using molecular markers for the mutant AHAS allele (ahas).
Backcrossing is repeated until BC3 to BC6
BC3-6 plants: 50% AHAS/ahas and 50% AHAS/ahas
The 50% AHAS/ahas are selected using molecular markers for the mutant AHAS allele (ahas). To reduce the number of backcrossings (e.g. until BC3 in stead of BC6), molecular markers can be used specific for the genetic background of the elite parent.
BC3-6 S1 cross: AHAS/ahas X AHAS/ahas
BC3-6 S1 plants: 25% AHAS/AHAS and 50% AHAS/ahas and 25% ahas/ahas
Plants containing ahas are selected using molecular markers for the mutant AHAS allele (AHAS). Individual BC3-6 S1 or BC3-6 S2 plants that are homozygous for the mutant AHAS allele (ahas/ahas) are selected using molecular markers for the mutant and the wild-type AHAS alleles. These plants are then used for seed production.

To select for plants comprising a point mutation in an AHAS allele, direct sequencing by standard sequencing techniques known in the art, such as those described in Example 1, can be used.

1. A plant comprising in its genome at least one mutant AHAS allele, said mutant AHAS allele being a full knockout AHAS allele.

2. (canceled)

3. The plant of claim 1, wherein said full knockout allele is selected from the group consisting of:
a) a nucleotide sequence comprising a stop codon at a position corresponding to nt 871-873 of SEQ ID NO: 1 or nt 826-828 of SEQ ID NO: 3;
b) a nucleotide sequence comprising a stop codon at a position corresponding to nt 862-864 of SEQ ID NO: 1 or nt 808-810 of SEQ ID NO: 5;
c) a nucleotide sequence comprising a stop codon at a position corresponding to nt 775-777 of SEQ ID NO: 1 or nt 721-723 of SEQ ID NO: 5; or
d) a nucleotide sequence comprising a stop codon at a position corresponding to nt 799-801 of SEQ ID NO: 1 or nt 745-747 of SEQ ID NO: 5.

4. The plant of claim 1, further comprising in its genome at least one second mutant AHAS allele, said second mutant AHAS allele encoding a herbicide tolerant AHAS protein.

5-9. (canceled)

10. A plant cell, seed, or progeny of the plant of claim 1.

11. A Brassica seed selected from the group consisting of:
a) Brassica seed comprising AHAS1-HETO112 having been deposited at the NCIMB Limited on Dec. 17, 2009, under accession number NCIMB 41690;
b) Brassica seed comprising AHAS3-HETO102 having been deposited at the NCIMB Limited on Dec. 17, 2009, under accession number NCIMB 41687;
c) Brassica seed comprising AHAS3-HETO103 having been deposited at the NCIMB Limited on Dec. 17, 2009, under accession number NCIMB 41688; or
d) Brassica seed comprising AHAS3-HETO104 having been deposited at the NCIMB Limited on Dec. 17, 2009, under accession number NCIMB 41689.

12. A Brassica plant, or a cell, part, seed or progeny thereof, obtained from the seed of claim 11.

13. A nucleic acid molecule encoding a full knockout AHAS allele.

14. (canceled)

15. The nucleic acid molecule of claim 13, wherein said nucleotide sequence is selected from the group consisting of:
a) a nucleotide sequence comprising a stop codon at a position corresponding to nt 871-873 of SEQ ID NO: 1 or nt 826-828 of SEQ ID NO: 3;
b) a nucleotide sequence comprising a stop codon at a position corresponding to nt 862-864 of SEQ ID NO: 1 or nt 808-810 of SEQ ID NO: 5;
c) a nucleotide sequence comprising a stop codon at a position corresponding to nt 775-777 of SEQ ID NO: 1 or nt 721-723 of SEQ ID NO: 5; or
d) a nucleotide sequence comprising a stop codon at a position corresponding to nt 799-801 of SEQ ID NO: 1 or nt 745-747 of SEQ ID NO: 5.

16-17. (canceled)

18. A method for combining a full knockout AHAS allele of claim 13 with a herbicide tolerant AHAS allele in one plant comprising the steps of:
a) generating and/or identifying at least one plant comprising at least one selected full knockout AHAS allele and at least one plant comprising at least one selected herbicide tolerant AHAS allele;
b) crossing the at least two plants and collecting F1 hybrid seeds from the at least one cross; and
c) optionally, identifying an F1 plant comprising at least one selected full knockout AHAS allele and the at least one selected herbicide tolerant AHAS allele.

19. A method for producing a plant of claim 4 comprising combining mutant AHAS alleles in or to one plant, according to claim 18.

20. A method to increase the herbicide tolerance of a plant comprising combining at least one full knockout AHAS allele and at least one herbicide tolerant AHAS allele in the genomic DNA of said plant.

21-22. (canceled)

23. A method for controlling weeds in the vicinity of crop plants, comprising the steps of:
a) planting in a field seeds produced by the plant of claim 4; and
b) an effective amount of AHAS-inhibiting herbicide to the weeds and to the crop plants in the field to control the weeds.

24. The method of claim 23, further comprising prior to step a) the step of applying an effective amount of AHAS-inhibiting herbicide to said field.

25. A method for treating a plant of claim 4, characterized in that said plants are treated with one or more AHAS-inhibiting herbicides.

26. (canceled)

27. The method of claim 23, wherein said AHAS-inhibiting herbicide is methyl 4-[(4,5-dihydro-3-methoxy-4-methyl-5-oxo-1H-1,2,4-triazol-1-yl)carbonylsulfamoyl]-5-methylthiophene-3-carboxylate.

28. The method of claim 23, wherein said plant is tolerant to an application of at least 5.0 g a.i./ha of methyl 4-[(4,5-dihydro-3-methoxy-4-methyl-5-oxo-1H-1,2,4-triazol-1-yl)carbonylsulfamoyl]-5-methylthiophene-3-carboxylate.

29. The method of claim 23, wherein said plant is selected from the group consisting of B. juncea, B. napus, B. rapa, B. carinata, B. oleracea and B. nigra.

30. Use of a full knockout AHAS allele of claim 13 to obtain a herbicide tolerant plant.

31. Use of the plant of claim 1 to produce seed comprising one or more full knockout AHAS alleles.

32. Use of the plant of claim 1 to produce a crop of oilseed rape, comprising one or more full knockout AHAS alleles.


SEQUENCE LISTING (6 sequences)

SEQ ID NO: 1 - DNA, 2010 bp, Arabidopsis thaliana
  CDS (1)..(2010)
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtgacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatggcaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatac

SEQ ID NO: 2 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWQDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 3 - DNA, 1965 bp, Brassica napus
  CDS (1)..(1965)
atggcggcggcaacatcgtcttctccgatctccttaaccgctaaaccttcttccaaatcccctctacccatttccagattctcccttcccttctccttaaccccacagaaagactcctcccgtctccaccgtcctctcgccatctccgccgttctcaactcacccgtcaatgtcgcacctccttcccctgaaaaaaccgacaagaacaagactttcgtctcccgctacgctcccgacgagccccgcaagggtgctgatatcctcgtcgaagccctcgagcgtcaaggcgtcgaaaccgtctttgcttatcccggaggtgcttccatggagatccaccaagccttgactcgctcctccaccatccgtaacgtccttccccgtcacgaacaaggaggagtcttcgccgccgagggttacgctcgttcctccggcaaaccgggaatctgcatagccacttcgggtcccggagctaccaacctcgtcagcgggttagcagacgcgatgcttgacagtgttcctcttgtcgccattacaggacaggtccctcgccggatgatcggtactgacgccttccaagagacaccaatcgttgaggtaacgaggtctattacgaaacataactatttggtgatggatgttgatgacatacctaggatcgttcaagaagctttctttctagctacttccggtagacccggaccggttttggttgatgttcctaaggatattcagcagcagcttgcgattcctaactgggatcaacctatgcgcttacctggctacatgtctaggttgcctcagcctccggaagtttctcagttaggtcagatcgttaggttgatctcggagtctaagaggcctgttttgtacgttggtggtggaagcttgaactcgagtgaagaactggggagatttgtcgagcttactgggatccccgttgcgagtactttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggctttcgctagcagggctaaaattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttccctttgagcttcaaaacgtttggagaagccattcctccgcagtacgcgattcagatcctcgacgagctaaccgaagggaaggcaattatcagtactggtgttggacagcatcagatgtgggcggcgcagttttacaagtacaggaagccgagacagtggctgtcgtcatcaggcctcggagctatgggttttggacttcctgctgcgattggagcgtctgtggcgaaccctgatgcgattgttgtggatattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacaccaggaccatacctgttggatgtgatatgtccgcaccaagaacatgtgttaccgatgatcccaagtggtggcactttcaaagatgtaataacagaaggggatggtcgcactaagtac

SEQ ID NO: 4 - protein, 655 aa, Brassica napus
MAAATSSSPISLTAKPSSKSPLPISRFSLPFSLTPQKDSSRLHRPLAISAVLNSPVNVAPPSPEKTDKNKTFVSRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQILDELTEGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 5 - DNA, 1956 bp, Brassica napus
  CDS (1)..(1956)
atggcggcggcaacatcgtcttctccgatctccttaaccgctaaaccttcttccaaatcccctctacccatttccagattctcccttcccttctccttaaccccacagaaaccctcctcccgtctccaccgtcctctcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgccttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtac

SEQ ID NO: 6 - protein, 652 aa, Brassica napus
MAAATSSSPISLTAKPSSKSPLPISRFSLPFSLTPQKPSSRLHRPLAISAVLNSPVNVAPEKTDKIKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY